(19) United States
(12) Patent Application Publication
(10) Pub. No.: US 2008/0318240 A1
(43) Pub. Date: Dec. 25, 2008

(54) METHODS FOR UTILIZING ESR COPY NUMBER CHANGES IN BREAST CANCER TREATMENTS AND PROGNOSES

(75) Inventors: Kirsten Vang Nielsen, Bronshoj (DK); Sven Muller, Lyngby (DK); Bent Laursen Ejlertsen, Bronshoj (DK)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Dako Denmark A/S

(21) Appl. No.: 12/128,967

(22) Filed: May 29, 2008

Related U.S. Application Data
(60) Provisional application No. 60/932,426, filed on May 31, 2007.
(60) Provisional application No. 61/028,534, filed on Feb. 14, 2008.

(30) Foreign Application Priority Data
May 16, 2008 (DK) PCT/DK2008/000184

(51) Int. Cl. C12Q 1/68

(52) U.S. Cl. 435/6

(57) ABSTRACT

The present invention relates to methods for estimation of efficacy of therapeutic treatment of cancer patients, in particular breast cancer patients. The estimation is based on determining of the status of aberration of the estrogen receptor alpha gene (ESR1) in situ, and, optionally, the status of aberration of a gene related to ESR1. In particular, the invention relates to determining the presence or absence and, if present, the type of aberration, e.g. amplification, duplication, polyploidization, deletion or translocation of the ESR1 gene in the tumor cells of the patient. The invention further relates to a kit-in-parts comprising probes for the determining the status of aberration of ESR1 and ESR1-related genes in situ.

This application claims priority to PCT International Application Number PCT/DK2008/000184, filed May 16, 2008, and the benefit of U.S. Provisional Application Nos. 60/932,426, filed May 31, 2007 and 61/028,534, filed Feb. 14, 2008, all of which are incorporated herein by reference.

### FIELD OF THE INVENTION

The present invention relates to methods for estimation of efficacy of therapeutic treatment of cancer patients, in particular breast cancer patients. The estimation is based on determining of the status of aberration of the estrogen receptor alpha gene (ESR1) in situ, and, optionally, the status of aberration of a gene related to ESR1. In particular, the invention relates to determining the presence or absence and, if present, the type of aberration, e.g. amplification, duplication, polyploidization, deletion or translocation of the ESR1 gene in the tumor cells of the patient. The invention further relates to a kit-in-parts comprising probes for the determining the status of aberration of ESR1 and ESR1-related genes in situ.

### BACKGROUND OF THE INVENTION

### Estrogen Receptor

The estrogen receptor (ER) has both predictive and prognostic utility and is the most widely used marker for clinical decisions in cancer, in particular breast cancer (see for review (Goldhirsch A, et al., Ann of Oncology, 16:15-69-1583, 2005). Using immunohistochemical (IHC) assays 70% to 85% of breast cancer patients will have estrogen receptor positive tumors depending on the cutoff used. Estrogenic effects are mediated by two forms of the estrogen receptor, ERα (referred herein as ER) and ERβ, although the function of ERβ still is unclear. Activity of both ER isoforms have been related to cancer (Shupnik, M. A., Piit, L. K., Soh, A. Y., Anderson, A., Lopes, M. B., Laws, E. R., Jr: Selective expression of estrogen receptor alpha and beta isoforms in human pituitary tumors. J. Clin. Endocr. Metab. 83:3965-3972, 1998; Skliris G P, Leygue E, Curtis-Snell L, Watson P H, Murphy L C. Expression of oestrogen receptor-beta in oestrogen receptor-alpha negative human breast tumours. Br J Cancer. 4; 95(5):616-26, 2006; Satake M, Sawai H, Go V L, Satake K, Reber H A, Hines O J, Eibl G. Estrogen receptors in pancreatic tumors. Pancreas. 33(2):119-27, 2006).

The effects of estrogens include proliferation and differentiation in reproductive tissue and have been linked to development and progression of breast cancer. Although the proportion of ER positive cells changes in the normal resting breast, only 15-25% of epithelial cells are ER positive and are for the most part non-dividing. Proliferation induced by estrogen mainly takes place in the ER negative cells surrounding the luminal epithelial cells. Dissimilarly, proliferation of ER positive epithelial cells in breast tumors is estrogen regulated. The mechanism behind the translation to ER dependency has not been clearly described.

The ER is encoded by the ESR1 gene localized on chromosome 6q25.1. One mechanism suggested to play a role in the progression of human breast cancer from hormone dependence to independence is the expression or altered expression of mutant and/or variant forms of the estrogen receptor. Two major types of variant ESR1 mRNA had been reported in human breast biopsy samples so far: truncated transcripts and exon-deleted transcripts. Larger-than-wildtype ESR1 mRNA RT-PCR products was detected in 9.4% of 212 human breast tumors analysed. Cloning and sequencing of these larger RT-PCR products showed 3 different types: complete duplication of exon 6 in 7.5%; complete duplication of exons 3 and 4 in 1 tumor; and a 69-bp (base pair) insertion between exons 5 and 6 in 3 tumors. Gross structural rearrangements of ESR1 were not identified in a series of 188 primary breast cancers using Southern hybridisation, and subsequent studies have confirmed that ESR1 translocations and copy number changes are uncommon in breast cancers. These observations may however reflect a low sensitivity of the applied technologies rather than the actual gene status of the examined samples as recently it has been reported that a copy number of ESR1 changes in breast cancer

Transcriptional activation is mediated by two activation domains (AF), AF-1 and AF-2. The AF-1 domain is located in the N-terminus of the receptor and has a ligand independent function that can be enhanced by phosphorylation in the mitogen-activated protein kinase (MAPK) pathway. The AF-2 domain has a ligand dependent function and is located in the ligand binding part in the C-terminus of the receptor.

### Coregulators

Gene activation requires the joint action of transcription factors and coactivators, and expression of coactivators is a substantial component of gene control. A major search for coactivators and corepressors was initiated in 1994 when interactions of a larger set of proteins in a ligand-dependent manner with the estrogen receptor was demonstrated. Despite the fact that many of the components have been identified, the manners leading to the exchange of these complexes by transcription factors is still unclear. Two separate models have been proposed. According to one model, distinct coactivator and corepressor complexes are supposed to be present in a preformed state are recruited to the chromatin by activation of the nuclear receptor. Another model suggests that coactivators and corepressors are present in the same complexes and just reorder for transcriptional activation. The exchange of coactivator and corepressor complexes by transcription factors is still unclear despite the identification of the components in these complexes. The coexistence in the complexes of coactivators and corepressors has been reported repeatedly, e.g. interaction between NCOA3 (AIB1), N-CoR and SMRT.

NCOA3 (AIB1) encodes nuclear receptor coactivator 3 which is mapped to 20q12. NCOA3 binds directly to nuclear receptors and stimulates the transcriptional activities in ligand-dependent fashion. The NCOA family including NCOA1, NCOA2, and NCOA3 are widely expressed and coactivate the majority of nuclear receptors including ER. NCOA3, also known as AIB1, pCIP, RAC3, SRC3, and ACTR, seems to have a dramatic impact on regulation in cancer, especially breast and prostate cancer (H Chen 1997). A high level of NCOA3 secondary to amplification has been found in breast cancers, hence the alias AIB1 (amplified in breast cancer 1). NCOA3 coactivates ERα to a larger extent than ERβ, and may antagonize the action of tamoxifen (J Font de Mora 2000). AIB1 or NCOA3 is not ER exclusive and inactivation of AIB1 by siRNAs reduces cancer growth. NCOA1 (SRC-1) was the first steroid coactivator cloned and its interaction with ER and PgR seems to be influenced by agonists and antagonists. NCOA2 (TIF2 or GRIP1) also interacts with steroid receptors in a ligand-dependent manner.

The molecular basis of the interactions between steroid receptors and corepressors is even more unclear than the interaction with coactivators. The nuclear receptor corepressor NCOR1 (N-CoR) and the silencing mediator for retinoid and thyroid hormone receptors NCOR2 (SMRT) were initially recognized as elements in the repression associated with un-liganded retinoic acid and thyroid hormone receptors. Low NCOR1 mRNA expression in the tumors of patients with ER positive primary breast has been associated with a significantly shorter relapse-free survival. NCOR1 was selected based on high-level amplification. NCOR2 is located on 12q24 and is structurally very similar to NCOR1, but does not seem to be amplified to the same levels as NCOR1. In addition to NCOR1 and NCOR2 corepressor activity has also been demonstrated for several other molecules including MTA, REA, RTA and NROB1 (DAX1).

Scaffold attachment factor B1 (SAFB/SAFB1/HET) and B2 (SAFB2/KIAA0138) resides closely on 19p13.3 and are essential for transcriptional regulation as well as numerous other cellular processes. A tumor-suppressor function might be expected as both mutations and large deletions of SAFB1 have been identified in breast cancers. SAFB expression is lost in around 20% of breast cancers and has been associated with a poor survival.

### Cancer Therapy Directed to Hormone Receptors

### Aromatase Inhibitors

Intratumoral aromatase activity in breast cancers could, especially in postmenopausal patients, represent the major source of estrogen which, in these tumors, maintains malignant growth. Intracellular concentrations of estradiol are more than 20-fold higher than in the plasma. Patients with high intratumoral aromatase content could therefore, in particular, benefit from treatment with aromatase inhibitors. A central dogma for extragonadal estrogen biosynthesis is that conversion of cholesterol to C₁₉ steroids only takes place in the adrenal cortex and ovaries. Circulating pro-hormones or C₁₉ precursors are present in the circulation of postmenopausal women at concentrations which are orders of magnitude greater than those of active sex steroids and include testosterone, androstenedione, dehydroepiandrosterone (DHEA), dehydroepiandrosterone sulfate (DHEAS). This large pool of precursors is accessible in peripheral tissues for conversion to estrogen. Ten patients, 7 of whom are women, with an inherited mutation in CYP19 have been reported. No effect of gender was observed in these patients from estrogen deprivation with respect to lipid and carbohydrate metabolism.

Aromatase inhibitors can be classified by mechanism of action and generation that essentially relate to potency and selectivity. The third generation (3G) aromatase inhibitors are potent and selective inhibitors of the aromatase enzyme. Anastrozole and letrozole are non-steroidal derivates of triazole and imidazole with a high but reversible binding capacity to the p450 domain of the aromatase enzyme. Exemestane is a steroidal compound that binds irreversibly to the substrate pocket and therefore has been named an aromatase inactivator. 17-hydroexemestane, the main metabolite of exemestane, has androgenic activity and suppresses sex-binding globulin in a dose-dependent manner. Direct measurements of the activity of aromatase inhibitors are hampered by lack of sensitivity of estrogen assays. Instead, a double-tracer injection of ³H-androstenedione and ¹⁴C-estrone with calculation of total body aromatization based on the isotope ratio of estrogen metabolites has been used. The double-tracer technique has revealed aromatase inhibition in the range of 50-90% from first and second-generation aromatase inhibitors and 98% or above for third generation compounds. When anastrozole and letrozole were compared in a small crossover study, letrozole led to a more substantial suppression of aromatase activity in all patients concurrently with a higher suppression of plasma estrogen levels. Clinically relevant differences therefore might exist even in-between third generation aromatase inhibitors.

The third generation (3G) aromatase inhibitors should be considered for first line endocrine therapy of hormone receptor positive metastatic breast cancer in postmenopausal breast cancer patients. Furthermore, 3G aromatase inhibitors should be used either in sequence with tamoxifen or alone in the adjuvant treatment of postmenopausal patients with hormone receptor positive breast cancer, and should also be considered when preoperative endocrine therapy is indicated.

Estrogen levels are excessive suppressed by the third-generation aromatase inhibitors, but preclinical studies suggests that breast cancer cells can become hypersensitive to estrogen in the absence or at low levels of estrogen. A further reduction in estrogen level, even from an ultra low point, could from a theoretical view be beneficial, and the therapeutic implications of COX inhibitors are under investigation in this setting.

### Estrogen Receptor Modulators

Over a period exceeding 30 years, tamoxifen has been shown to be an effective treatment not only in all aspects of hormone receptor positive invasive breast cancer (preoperatively, adjuvant and advanced) but also for ductal carcinoma in situ and for prevention of breast cancer. Since the early 1970s, tamoxifen has been an essential element of breast cancer therapy and remains the unchallenged standard adjuvant endocrine therapy in premenopausal patients with hormone receptor positive breast cancer. Until recently, tamoxifen was also the sole endocrine standard for adjuvant therapy in postmenopausal women with breast cancer but might be considered in sequence with an aromatase inhibitor.

### Progestins and Selective Steroid Sulfatase Inhibitors

Besides inhibition of the steroid sulfatase pathway, progestins have multiple cellular actions including receptor binding e.g. progesterone, androgen, and glucocorticoid receptors and loering estradiol, estrone, testosterone, androstenidione, adrenocorticotropic hormone and cortisol levels. Following Stoll's pivotal work in the mid 1960's, several trials were conducted using medroxyprogesterone acetate (MPA) and megestrol acetate (MA) in patients with metastatic breast cancer. Compilations of results from 16 trials, including 1342 patients, have demonstrated a 26% response rate (range 14-44%) and a comparable efficacy to tamoxifen and aromatase inhibitors has been demonstrated. Major weight gain and potentially life-threatening thromoboembolic events, however, clearly limit the use of MA and MPA.

### ER and PgR Assays

Endocrine treatments are currently recommended to breast cancer patients according to estrogen and progesterone receptor (PgR) status. To determine the status of the receptors, the assays discussed below are currently used.

Ligand-binding assays (LBA), such as the dextran-coated charcoal assay (DCC) were the first standardized ER assays and they have been validated on several occasions. LBA assays use tumor tissue frozen immediately after excision in liquid nitrogen. The tissue is pulverized in liquid nitrogen, and cytosols are prepared. A labeled ligand (e.g. ³H estradiol) allows quantization of ER content, and the addition of a second ligand allows a dual quantization of ER and PgR. LBAs require large amounts of fresh-frozen tissue leading to severe logistic complications. They are technically demanding, labor extensive and require radioactive reagents. LBAs are based on whole-tissue homogenates, and unavoidable differences in the ratio of benign and tumor cells limits their sensitivity and specificity.

Specific monoclonal ER antibodies were developed more than 25 years ago, and IHC techniques have several potential advantages over LBAs, especially the ability to differentiate between benign and tumor cells. Furthermore, IHC is technically less demanding, is safer, and applicable on a range of different samples including cell aspirates, frozen and paraffin embedded tissue and, consequently, less costly. Still, results of IHC have shown persistent variability, mainly due to the use of a variety of different laboratory protocols and antibodies (e.g.: H222, H226, D547, D75, 1D5), several often-arbitrary methods for scoring of the results and an overall lack of standardization.

The ASCO Tumor Marker Panel has acknowledged the prognostic value of ER and PgR based and the guidelines of both NIH and St. Gallen, which recommend their use as prognosticators. The primary use of primarily ER is however as a selection marker for endocrine therapy in the adjuvant and advanced setting of breast cancer.

A cutoff for ER positivity has never completely been agreed upon for LBA, probably due to methodological limitations. In some studies, responses to endocrine therapy have been observed in patients with ER levels as low as 4 to 10 fmol/mg protein using LBA. Others have used 10 fmol/mg as the lower cutoff. All studies that examined cutoff for ER utilized tamoxifen and other endocrine therapies as ovarian suppression and aromatase inhibitors may, for several reasons, be more efficient in patients with low levels of ER.

When converting from LBA to IHC, most laboratories have used an arbitrary cutoff of 10% or 20% positive tumor cells. This has been based on numerous studies finding an 89% to 90% agreement when comparing ER status in the same tumors, using both LBA and IHC. The Allred score categorizes IHC results according to both the proportion of stained cells and the intensity of the staining. Benefit from adjuvant tamoxifen has been demonstrated with Allred scores as low as 3 (corresponding to as few as 1% to 10% weakly positive cells), and most institutions would not offer these patients endocrine therapy.

It is not possible to exactly define a lower cutoff on ER for endocrine responsiveness in breast cancer patients using either LBA nor IHC. Both methods have low sensitivity and specificity in weakly positive tumors, and this may adversely affect treatment decisions.

### SUMMARY OF THE INVENTION

The present invention relates to novel methods for estimating the efficacy of the selected cancer therapy, selecting an efficient therapeutic treatment for a cancer patient, stratification of cancer patients for therapeutic treatment and estimating the risk of disease recurrence in cancer patients which have been or are under the course of hormone therapeutic treatment.

The methods of the invention involve determining the status of aberration of the ESR1 gene, and, optionally, the status of aberration of one or more genes related to ESR1, in a cancer patient, wherein the term “status of aberration” refers to the presence or absence of an aberration of the gene and, if an aberration is present, the type of the aberration, e.g. amplification, duplication, polyploidization, deletion or translocation of the ESR1 gene in situ in the tumor cells of the patient. The determined status of aberration of the ESR1 gene and, optionally, an ESR1-related gene, is used as a prognostic factor of efficacy of hormone or combined cancer therapy (hormone in combination with chemotherapy).

The invention is based on an unexpected finding that the presence or absence of an aberration of the ESR1 gene, in particular amplification of the ESR1 gene in situ in a patient (the term “patient” is interchangeably used herein with the term “subject” or “cancer patient”), makes the this patient non-responsive to a hormone therapy, although, said cancer patient may still benefit from an alternative chemotherapeutic treatment.

Further, it was unexpectedly found, that aberration of ESR1 in cancer patients often correlates with aberration of some ESR1-related genes. The term “ESR1-related genes” in the present context refers to genes that have a genetic connection to the ESR1 gene, e.g. genes located in the same chromosome locus, or regulatory connection, e.g. genes involved in regulation of activity of ESR1 or activity of the ESR1-related products, i.e. RNA and proteins. A gene related to the ESR1 gene may be selected from, but not limited to the genes encoding nuclear receptor coactivators (NCOA1, NCOA2, and NCOA3), the nuclear receptor co-repressor NCOR1 (N-CoR), scaffold attachment factors B1 and B2, the silencing mediator for retinoid and thyroid hormone receptors NCOR2 (SMRT), progesterone receptor (PGR), HER2 (ERBB2). Exemplary genes, which status may further or additionally be determined, may be selected from the genes involved in estrogen synthesis, nuclear receptors and cofactors. Non-limited examples of these genes are discussed below. The ESR1-related gene may be selected from, but not limited to PGR, SCUBE2, BCL2, BIRC5, PTGS2 and FASN. Thus, according to the invention, determining the status of aberration of ESR1 in some embodiments may optionally be supplemented by determining the status of aberration of one or more ESR1-related gene. Such determination is optional, as determining the status of aberration of ESR1 may be sufficient for the prognosis. However, prognosis based on the data on aberration of ESR1 and one or more ESR1-related genes in situ may be more valuable.

According to the invention detection of amplified ESR1 and, optionally, amplified one or more the ESR1-related genes in situ is correlated with poor outcome of hormonal therapy in a cancer patient who has these genes amplified, and thus may serve as a valuable tool for predicting hormone therapy resistance. Deletion of ESR1 may be indicative of that the hormonal therapy is not optimal treatment for the patient neither, whereas the absence of aberration of ESR1, i.e. normal ESR1, may be an indicator of success of hormonal treatment of the patient. Thus, the patients may be stratified for a particular treatment based of the determined status of aberration of ESR1 and, optionally, one or more ESR1-related genes. Amplification of any or all of the latter genes may also used for prediction of the outcome of a combined hormone and chemotherapeutic therapy.

The methods of the invention advantageously expand approaches currently used in the art for the same purposes. The methods of the invention can be used alone, i.e. not supplemented by any additional testing currently used for same purposes, i.e. for selecting an efficient therapeutic treatment of a cancer patient, estimating the efficacy of the selected therapeutic treatment, stratifying patients for different therapy, or they can be used in combination with any additional testing based on similar or different approaches currently employed in the field.

The invention also relates to compositions, e.g a kit-in-parts, useful for determining an aberration of the above mentioned genes in situ in an in vitro assay.

### BRIEF DESCRIPTION OF THE DRAWINGS

FIG. 1 demonstrates the general design of the FISH probe mix for detection of the ESR1 gene copy number in situ. The probe is constructed as a mixture of Texas Red and Fluorescein labeled probes in which the red BAC (Bacterial Artificial Chromosome) DNA based probe is specific for the ESR1 gene at 6q25 and the green PNA (Peptide Nucleic Acid) based reference probe is specific for the centromeric region of chromosome 6.

FIG. 2 demonstrates the positions at chromosome 6q25 of the BAC clones used for construction of the ESR1 probe (marked in rectangles), relative to the position of the genomic ESR1 sequence (marked with an arrow).

FIG. 3 presents the results of a FISH analysis showing the specific hybridization of the ESR1 probe mixture to 6q25 (four red signals—indicated by four solid arrows) and the centromeric region of chromosome 6 (two green signals—indicated by two dotted arrows) to normal human metaphase spreads. The red signals are indicated by a solid arrow, the green signals are indicated by a dashed arrow.

FIG. 4 shows the results of a FISH testing of ESR1/CEN-6 FISH Probe Mix on a mamma carcinoma FFPE tissue with ESR1 deletion. A=ESR1 probe (Texas Red filter); B=CEN-6 probe (Fluorescein filter); C=DAPI counterstain (DAPI filter); D=ESR1/CEN-6 FISH Probe Mix in triple filter

FIG. 5 shows that the tamoxifen treated patients who had tumors containing amplification in any of BCL2, SCUBE2, PGR, BIRC5, COX2 (five genes panel I) also had a worse outcome of the treatment (judged as recurrence of the disease) compared to tamoxifen treated patients with tumors not containing any amplification in these 5 genes. The p-value is 0.0001.

FIG. 6 shows that the tamoxifen treated patients who had tumors containing amplification in BCL2, SCUBE2, PGR, BIRC5, COX and ESR1 (six genes panel) also had a worse outcome of the treatment (judged as recurrence of the disease) compared to tamoxifen treated patients with tumors not containing amplification in any of these 6 genes. The p-value is 0.0001.

FIG. 7 shows that the tamoxifen treated patients who had tumors containing amplification in any of BCL2, SCUBE2, PGR, BIRC5, FASN, ERS2 and ESR1 (seven genes panel) also had a worse outcome of the treatment (judged as recurrence of the disease) compared to tamoxifen treated patients with tumors not containing amplification in any of the 7 genes. The p-value is 0.0001.

FIG. 8 shows that amplifications of the genes of the five genes panel II, ESR1, PGR, SCUBE2, BCL2, and BIRC5, are associated with a higher likelihood of recurrence of the disease and worse outcome of tamoxifen treatment. The p-value is 0.0001.

FIG. 9 shows that tamoxifen treated patients who had tumors containing amplification in any of BCL2, SCUBE2, BIRC5 and ESR1 (four genes panel) have a worse outcome compared to tamoxifen treated patients with tumors not containing amplification in any of the 4 genes. The p-value is 0.0001.

### DETAILED DESCRIPTION OF THE INVENTION

The present invention provides new methods relating to prognostic value of copy number changes of the ESR1 and a group of ESR1-related genes in cancer, e.g. breast cancer (the term “ESR1 gene” is interchangeably used herein with the term “ESR1” or “estrogen receptor gene”; the term “ESR1-related genes” refers to genes that have a genetic connection to the ESR1 gene, e.g. genes located in the same chromosome locus, or regulatory connection, e.g. genes involved in regulation of activity of ESR1 or activity of the ESR1-related products, i.e. RNA and proteins.). The methods of the invention are useful for estimating the efficiency of cancer therapy, in particular breast cancer therapy, for stratification of cancer patients for different therapy, for estimating the likelihood of recurrence of the disease in patients who have been or is under treatment with a hormonal therapy.

The methods of the invention involve determining the status of ESR1 aberration and optionally the status of aberration of at least one of the ESR1-related genes, e.g. ESR2, COX, BCL2, SCUBE2, PGR, BIRC5, FASN, wherein the determined status is indicative of whether a selected therapeutic treatment will be efficient for a cancer patient or not. Cancer patients, for whom the status of aberration of the ESR1 gene and at least one of the ESR1-related genes, has been determined, may be stratified based on this status for different cancer therapy.

By the term “gene aberration” is meant any change in the DNA sequence of a gene or a change in a sequence/region related to a gene, e.g. a regulatory chromosomal region of the gene. The term “gene” in the present context means the unit of inheritance that occupies a specific locus on a chromosome, which includes regulatory regions, transcribed regions and/or other regions having other functional activities. Preferable gene aberrations may be selected but not limited to amplifications, duplications, polyploidization, deletions and/or translocations of the full-length DNA sequence of the gene, fragments/parts of the gene DNA sequence and/or gene-related DNA sequences in the subject genome or fragments/parts of said DNA sequences, or of the full-length gene with flanking regions (also known as an amplicon). Gene aberrations may include increased copy number of the chromosome harboring the gene of interest.

The term “status of an aberration of a gene” refers to the presence or absence of an aberration of a gene in a subject genome and, if an aberration is present, the type of aberration, e.g. amplification, duplication, polyploidization, deletion or translocation of the ESR1 gene in situ in a tissue sample obtained form the patient. When an aberration is absent, the gene is normal, i.e. the gene presents in the chromosomal DNA in a normal number of copies, the number of copies which normally comprise the genomic DNA located in the normal chromosomal position. The term “normally” in the present context relates to a subject who does not have or is not suspected of having cancer, in particular breast cancer. The status when aberration of a gene(s) of interest in a subject genome is absent is referred herein as normal gene. Amplification or deletion of a gene is reflected by the presence of increased or decreased number of copies of the gene in a subject genome, i.e. increased in case of amplification (or duplication or polyploidization) and decreased in case of deletion. The status when a gene of interest is present in a subject genome in an increased number of copies is referred herein as gene amplification, and when the gene is present in a subject genome in a decreased number of copies is referred herein as gene deletion. Further, the gene may be moved to another position by translocation. The gene may also be spit in two or more parts by translocation of a part of the gene.

The term “genome” refers to the total set of genes carried by an individual or cell.

A sequence/gene/region wherein the status of aberration is to be determined, is termed herein as “target sequence/gene/region” or “sequence/gene/region of interest”.

Determining the status of aberration of the gene of interest is preferably performed by using a gene analysis, wherein the term “gene analysis” means any analysis that may be suitable for analyzing genes, e.g. in situ hybridization, RT-PCR, sequencing, Southern blotting, CGH, and array CGH. In some preferred embodiments, the status of aberration of ESR1 and, optionally, at least one ERS1-related gene is determined in vitro, by an in situ hybridization analysis.

To perform a gene analysis, in particular in situ hybridization, various probes may be used.

“Probe” as used herein means any molecule or composition of molecules that may bind to the region(s)/sequence(s) related to the gene to be detected or visualized.

The invention in different embodiments relates to different types of probes, e.g. in some embodiments, the invention relates to specific probes.

“Specific probe” means any probe capable of binding specifically to regions to be detected, e.g. a genomic sequence related to the gene for which the status of aberration is to be determined, or a sequence of the gene product, such as protein or RNA molecule (non-limited examples of specific probes are described below).

In another embodiment, the invention relates to blocking probes.

“Blocking probe” means any probe capable of blocking, suppressing or preventing the interaction of a region to be detected with other probes or molecules.

The origin of probes of the invention may, in different embodiments, also be different, e.g. in some embodiments, it may be nucleic acid probes.

“Nucleic acid probe” means any molecule consisting of naturally occurring nucleobases. Preferably, the nucleobases on a nucleic acid probe of the invention are connected to each other and form a nucleobase sequence. It may be a nucleobase sequence-containing probe represented by an oligomer or polymer molecule comprising solely nucleotides, or analogs thereof, wherein said nucleotides are single elements, monomers, bound to each other so that they form a sequence of nucleotides; “nucleotide” as used herein, means any of several compounds that consist of a ribose or deoxyribose sugar joined to a purine or pyrimidine base and to a phosphate group; “oligomer” as used herein, means a sequence of 3-50 monomers, e.g. nucleotides, nucleobases; “polymer” as used herein, means a sequence of more than 50 monomers, e.g. nucleotides, nucleobases. Nucleic acid probes of the invention may be made of naturally occurring nucleic acid molecules, such as oligodeoxynucleic acids (e.g. DNA), oligoribonucleic acids (e.g. RNA, mRNA, siRNA), or fragments thereof.

In another embodiment a probe may be a nucleic acid analog probe.

The term “nucleic acid analog probe” refers to any molecule that is not a naturally occurring nucleic acid molecule or to any molecule that comprises at least one modified nucleotide, or subunit derived directly from a modification of a nucleotide. An example of nucleic acid analog probes may be probes comprising sequences of PNA, wherein “PNA” is the abbreviation of peptide nucleic acid. PNA's backbone is composed of repeating N-(2-aminoethyl)-glycine units linked by peptide bonds. The various purine and pyrimidine bases are linked to the backbone by methylene carbonyl bonds. PNAs are depicted like peptides, with the N-terminus at the first (left) position and the C-terminus at the right. Since the backbone of PNA contains no charged phosphate groups, the binding between PNA/DNA strands is stronger than between DNA/DNA strands due to the lack of electrostatic repulsion. Another non-limiting example of a modified naturally occurring molecule may be Locked Nucleic Acid (LNA). LNA is a modified RNA nucleotide. Ribose moiety of LNA nucleotide is modified with an extra bridge connecting 2′ and 4′ carbons. The bridge “locks” the ribose in 3′-endo structural conformation, which is often found in A-form of DNA or RNA. LNA nucleotides can be mixed with DNA or RNA bases in the oligonucleotide whenever desired.

Still, in another embodiment, a probe may be a peptide or protein probe.

Peptide and protein probes may be represented by full-length proteins or fragments thereof. Non-limiting examples of such proteins are antibodies, receptors, ligands, growth factors, DNA binding proteins. Peptide and protein probes may be prepared using recombinant technologies or synthetically, e.g. by using chemical synthesis. Peptide probes are usually shorter than protein probes and may comprise both natural and unnatural amino acids residues.

The principles of designing of probes capable of recognizing and specifically binding to genomic sequences are well known in the art: they can be found in a number of text books, e.g. Sambrook J., and Russel. D. W. Molecular Cloning: A Laboratory Manual, CSHL 3^{rd} ed, Cell Press, 2001. Techniques for preparation of different types of probes (probes of the invention) are also well known. The probes can also be designed and prepared on a request by a number of available commercial manufacturers.

All, nucleic acid, nucleic acid analog and protein probes, may bind a region of interest in situ in a in vitro assay. The probes may have any length suitable for detecting a target region, e.g. the full length gene sequence with flanking regions, the amplicon, within the gene of interest, or a reference sequence, e.g. a sequence of the centromeric region. A probe may consist of one individual sequence or nucleotides, amino acid residues or other monomers, representing thus a single probe. Such probe may be represented by a relatively long sequence and span up to 2 megabases (Mb). However shorter nucleotide sequences from about 0.5 kilobases (kb) to about 50 kb may be also used. A probe may comprise several individual probes, e.g. it is made up of small fragments of nucleotide sequences of varying sizes (e.g. from about 50 bp [base pairs] to about 500 bp each) such that the probe will in total span about 30 kb to about 2 Mb. The sequence of a nucleic acid or nucleic acid analog probe may comprise both regions of unique sequences and regions of repeated sequences. If such repeated sequences are undesirable in the probe sequence, they can be removed or blocked, for example by using blocking probes.

Nucleic acid analogue probes, like PNA probes, are usually shorter than nucleic acid probes, and they have well defined sequences. PNA probes typically comprise from about 10 to about 25 nucleobases. A PNA probe is usually composed of several individual PNA molecules, each having 10 to 25 nucleobase units.

Nucleic acid probes, nucleic acid analogue probes and protein probes may be employed in separate analyses or in combination in the same analysis. For example, in one testing, one set nucleic acid probes may be employed for detection of the sequence of interest and another set of probes comprising nucleic acid, nucleic acid analogue and/or protein probes may be employed for detection of the reference sequence or a product of the reference gene, such as a protein or RNA.

Probes may be and in some embodiments are preferably labeled.

Labeling of the probes may be done by using any well-known in the art methods, e.g. by means of enzymatic or chemical processes. Any labeling method known to those in the art can be used for labeling probes for the purposes of this invention, e.g. combined use of DNase I and DNA polymerase I for cutting DNA and labelled monomer insertion, also known as Nick Translation in case of DNA and e.g. chemical modification of amino derivatised oligo nucleotides or analogues in case of PNA.

The probes may bind to a sequence of the target gene, or a reference sequence, and hybridize under stringent conditions. Those of ordinary skill in the art of hybridization will recognize that factors commonly used to impose or control stringency of hybridization include formamide concentration (or other chemical denaturant reagent), salt concentration (i.e., ionic strength), hybridization temperature, detergent concentration, pH and the presence or absence of chaotropes. Optimal stringency for a probe/marker sequence combination is often found by the well-known technique of fixing several of the aforementioned stringency factors and then determining the effect of varying a single stringency factor. The same stringency factors can be modulated to thereby control the stringency of hybridization of a PNA to a nucleic acid, except that the hybridization of a PNA is fairly independent of ionic strength. Optimal stringency for an assay may be experimentally determined by examination of each stringency factor until the desired degree of discrimination is achieved. Generally, the more closely related the background causing nucleic acid contaminates are to the target sequence, the more carefully stringency must be controlled. Suitable hybridization conditions will thus comprise conditions under which the desired degree of discrimination is achieved such that an assay generates an accurate (within the tolerance desired for the assay) and reproducible result. Nevertheless, aided by no more than routine experimentation and the disclosure provided herein, those of skill in the art will easily be able to determine suitable hybridization conditions for performing assays utilizing the methods and compositions described herein.

Non-limiting examples of stringent conditions are described in the experimental procedure below and further non-limiting examples may be found in chapter 11 in Peptide Nucleic Acids, Protocols and Applications, Second Ed. Editor Peter E Nielsen, Horizon Scientific Press, 2003.

As discussed above one aspect of the invention relates to determining the status of aberration of ESR1 and optionally the status of aberration of at least one ESR1-related gene. The status of aberration may be determined in relation to a genomic reference sequence.

By the term “genomic reference sequence” is meant a sequence in situ which is not identical with the gene/sequence/region of interest. By applying a reference sequence located on the same chromosome as a gene of interest, the specific ploidy level of the given chromosome is decisive of whether a genomic target sequence (a sequence, the status of aberration of which is to be determined) will be found amplified, deleted, duplicated, translocated or normal.

The probe binding to a reference sequence may be targeted against the centromeric region of a chromosome where the gene of interest is located. Both nucleic acid probes, nucleic acid analogue probes as well as protein probes may be employed as reference probes. In spite of the great homology in the centromeric DNA of all human chromosomes, unique sequences have been identified and clones containing human chromosome specific centromeric repeat sequences have been constructed for the majority of human chromosomes for use as the reference sequences in situ hybridization assays. The length of a reference probe may be dramatically reduced without reduction of the signal intensity when probes targeted against centromeric repeat sequences are used. The advantage of using centromeric reference probes is that they do not contribute to background staining as they do not contain short and interspersed elements (SINEs and LINEs respectively).

Centromeric regions, e.g. the centromeric region of the chromosome where the a gene of interest is located or the centromeric region of another chromosome, can be specifically identified by in situ hybridization probes derived from clone centromeric sequences. These clone sequences may be used as reference probes. However, synthetic PNA probes may be preferred for centromer detection in situ. A useful PNA probe for detection of centromeric region is made of 10-25 bases. Some non-limited examples of centromeric regions reference probes are described below.

To measure the ploidy level of the cancer cells, the centromeric of any chromosome may be used. The chromosome that has least frequently undergone changes in breast cancer is chromosome 2 (Mitelman). Therefore, the centromeric of chromosome 2 would be useful as a general reference probe in breast cancer, regardless of the location of the gene of interest.

A locus specific probe (LSP) may be used as an alternative reference probe. Such probe are preferably targeted to the opposite chromosome arm than the arm of the gene of interest, to eliminate errors of the analysis originating in case whole arm deletions occurs. The LSP reference probe should not be placed in a region that has any relation to genome aberrations in cancer.

A number of gene analyses known in the art where the probes described above may be used for the purposes of the invention.

Fluorescence in situ hybridization (FISH) is an important tool for determining the number, size and/or location of specific DNA sequences in cells and may be applied in methods of the invention. Typically, the hybridization reaction where probes comprise a fluorescent label fluorescently stains the target sequences in situ so that their location, size and/or number can be determined using fluorescence microscopy, Ligth cycler, tacman, flow cytometry or any other instrumentation suitable for detection of fluorescence. DNA sequences ranging from whole genomes down to several kilobases can be studied using current in situ hybridization techniques in combination with commercially available instrumentation. In Comparative Genomic Hybridization (CGH) whole genomes are stained and compared to normal reference genomes for the detection of regions with aberrant copy number. In the m-FISH technique (multi color FISH), each separate normal chromosome is stained by a separate color (Eils et al, Cytogenetics Cell Genet 82: 160-71 (1998)). When used on abnormal material, the probes will stain the aberrant chromosomes thereby deducing the normal chromosomes from which they are derived (Macville M et al., Histochem Cell Biol. 108: 299-305 (1997)). FISH-based staining is sufficiently distinct such that the hybridization signals can be seen both in metaphase spreads and in interphase nuclei. Single and multicolor FISH, using nucleic acid probes, have been applied to different clinical applications, including prenatal diagnosis, leukemia diagnosis, and tumor cytogenetics, and is generally known as molecular cytogenetics.

Other gene analysis methods which may also be used for the purposes of the present invention is Real-Time PCR (RT-PCR), array CGH and Chromogenic In Situ Hybridization (CISH). Combination of any of these techniques is also applicable. In particular, a combination of FISH and CISH may be used, e.g. one probe may be labeled with a fluorescent label and another with a chromogen label so as to enable separate or simultaneous detection of the FISH signal and CISH signals.

According to the invention, the gene probe and the reference probe should be labeled differently, e.g. with labels which generate different colors such as e.g. red and green, respectively. Non-limiting examples of such labels may be fluorescent labels, such as Texas Red and Fluorescein. The blue DAPI color may be used for counterstaining to assist tissue localization and identification. Availability of control Hematoxylin-Eosin cut section may also be useful.

A gene analysis is preferably performed using a tissue sample obtained from a patient, e.g. a biopsy sample. The simplest way to perform the in situ hybridization analysis may be to cut the relevant number of sections from paraffin embedded tissue and hybridize a probe to each section. Alternatively, frozen tissue can be used or imprints. Hybridization demands only standard conditions. For most probes an internal reference, such as e.g. a centromeric probe, preferably should be included.

The status of an aberration of the gene may be measured as the actual number of copies of the sequence of interest present in the sample, e.g. number of copies of the gene, i.e. number of copies of ESR1 gene and/or copies of the ESR1-related genes of the invention.

In some embodiments, the status of an aberration of the gene may be determined as the actual amount of a gene product in the sample, e.g. total amount of the corresponding RNA or protein. In other embodiments, the status of an aberration of the gene may be defined as a ratio, where the amount of the sequence of interest is correlated to the amount of a reference sequence. In some embodiments it is preferred to use the latter evaluation. In other embodiments, the status of a gene aberration may be referred to cut-off values.

In other embodiments, the status of aberration of the gene may be determined using a combination of in vitro analysis of the status of the gene in situ and analysis of the gene products in a sample, e.g. by a combination of FISH and IHC or CISH and IHC, or FISH/CISH and evaluation of the levels of expression of one or more gene products.

For example, in a normal cell, two copies of each of the ESR1 genes are present. Theoretically, two signals derived from the probe bound to the complementary DNA strands should be visible. However, in some embodiments, in a sample prepared for performing gene analysis by in situ hybridization, due to cutting of sections from paraffin embedded tissue, whole nuclei may not be present. Therefore, a difference between theoretical and actual number of signals may be observed and cut-off values between normal and abnormal number of signals per cell will have to be determined empirically. Using a reference probe, two reference probe signals should be seen in a normal cell, and theoretically, the ratio between signals from gene probe and reference probe should be 1 (one). However, due to technical, biological and statistical reasons this absolute value is determined as a range, e.g. such as a range between 0.8 and 2.0, as, for example, in the case of HER2 FISH (package insert, Dako HER2 FISH pharmDx™ kit, code K5331). The FISH assay can be performed with and without one or more reference probes. Without a reference probe, only signals in one color from the target gene probe are scored, and the cut-off value between normal and amplified gene sequence is more than 3, preferably 4 or 5, although the theoretical value is 2. However, deletions cannot be scored in an assay without a reference probe or a reference sample.

A FISH assay may include one or more reference probes in addition to the gene probe, e.g. the ESR1 gene probe and centromeric probe labeled differently, e.g. with different fluorescent labels. The gene copy number may then be calculated by using the reference probe. Signals from each gene copy and signals from the corresponding reference sequences are detected and the ratio is calculated. As already mentioned, the reference sequence is a measure of the ploidy level, thus it indicates the number of chromosome copies. The most accepted cut-off value of a normal gene copy number is indicated by a ratio between 0.8 and 2.0. Gene deletion is indicated by a ratio below 0.8, whereas gene amplification is indicated by a ratio≧2.0.

The cut-off value of a normal gene copy number may also be established from a analyzing a normal material, i.e. a sample obtained from a control individual. Therefore alternative cut-off levels for a normal sample could be 0.93-1.19 or 0.8-1.6. Thus, the cut-off discriminating between deletion and normal ratio can be from 0.8 to 0.96 while the cut-off discriminating between normal and amplification can be from 1.19 to 2.0.

According to the invention, a cut-off value between 0.8 and 2 is indicative of a normal gene copy number and is predictive of better recurrence-free survival or overall survival of a patient predicting efficacy of hormonal therapy for the patient, whereas the presence of an aberration of the gene, reflected by a decreased (a cut-off value less than 0.8) or increased gene copy number (a cut-off value more than 2) is predictive of a worse prognosis, such as a worse recurrence-free survival or overall survival of a patient having a course of hormonal therapy.

Thus, the defined status of an aberration of the gene is correlated to the condition of interest, i.e. disease, in particular breast cancer, and to a response of the condition to a therapy. Thus, it may therefore be used for predicting the outcome of treatment, development of the disease and estimation of efficacy of therapeutic treatment.

Prognostic value of the determined status of aberration of the ESR1 gene and some of the ESR1-related genes is illustrated herein by non-limiting examples (see EXAMPLES).

### EMBODIMENTS OF THE INVENTION

In one embodiment the invention relates to a method for predicting the efficacy of a therapeutic treatment of a cancer patient comprising

determining the status of aberration of the estrogen receptor gene (ESR1) in a sample obtained from said patient; and

predicting the efficiency of a therapeutic treatment based on the determined status of aberration of the estrogen receptor gene (ESR1) in the sample obtained from the patient.

In another embodiment the invention relates to is a method for selecting a therapeutic treatment for a cancer patient comprising

determining the status of aberration of the estrogen receptor gene (ESR1) in a sample obtained from said patient; and

selecting a cancer therapy which is likely to be efficient for the patient based on the determined status of aberration of the estrogen receptor gene (ESR1) in the sample obtained from said patient.

In another embodiment of the invention relates to a method for stratifying cancer patients for therapeutic treatments comprising

determining a status of aberration of the estrogen receptor gene (ESR1) in samples obtained from the cancer patients; and

stratifying the cancer patients for different therapeutic treatments, wherein the selection of said therapeutic treatment is based on the determined status of aberration of the estrogen receptor gene (ESR1) in samples of the cancer patients.

In another embodiment the invention relates to a method for predicting disease recurrence in a cancer patient comprising

determining a status of aberration of the estrogen receptor gene (ESR1) in samples obtained from the cancer patients; and

predicting the disease recurrence in the cancer patient based on the determined status of aberration of the estrogen receptor gene (ESR1).

All the above methods comprise a step of genetic analysis of a sample obtained from a cancer patient in order to determine the status of aberration of ESR1. A method for genetic analysis may be any suitable method for analysis of genes in situ, e.g. one of the methods described above.

As discussed above, amplification of ESR1 alone is indicative of a poor outcome of hormone therapy in patients having ESR1 amplified. However, it was surprisingly found that amplification of ESR1 is often associated with amplification of some other genes related to the oestrogen metabolism, such as, e.g., PGR, ESR2, SCUBE2, BCL2, BIRC5, PTGS2 and/or FASN (termed herein as “ESR1-related genes”). Thus determining the status of aberration of these genes in addition to determining the status of aberration of ESR1 would be beneficial for more reliable stratification of cancer patients for a particular treatment or prognosis of the outcome of the treatment, e.g. prediction of the likelihood of recurrence of the disease. Accordingly, the methods of the invention in another embodiment further comprise a step of determining the status of aberration of an ESR1-related gene. As already mentioned above, the term “ESR1-related gene” in the present context refers to genes that have a genetic connection to ESR1, e.g. genes located in the same chromosome locus, or regulatory connection, e.g. genes involved in regulation of activity of ESR1 or activity of the ESR1-related products, i.e. RNA and proteins. A gene related to ESR1 may be selected from, but not limited to the genes encoding nuclear receptor coactivators (NCOA1, NCOA2, and NCOA3), the nuclear receptor corepressor NCOR1 (N-CoR), scaffold attachment factors B1 and B2, the silencing mediator for retinoid and thyroid hormone receptors NCOR2 (SMRT), progesterone receptor (PGR), HER2 (ERBB2). Exemplary genes, which status may further or additionally be determined, may be selected from the genes involved in estrogen synthesis, nuclear receptors and cofactors. Non-limited examples of these genes are shown in Table 1 below. Some preferred ESR1-related genes may be PGR, ESR2, SCUBE2, BCL2, BIRC5, PTGS2, COX and FASN, however, the invention is not limited the latter genes.

TABLE 1

| Gene | Position | Alias | Function | OMIM |
|---|---|---|---|---|
| PGR | 11q22.1 | PR | Progesterone receptor | 607311 |
| ESR2 | 14q23 | ER beta | Nuclear hormone receptor | 601663 |
| NCOA1 | 2p23 | SCR1 | Nuclear receptor coactivator 1 | 602691 |
| NCOA2 | 8q13.3 | GRIP1 | Nuclear receptor coactivator 2 | 601993 |
| | | TIF2 | | |
| NCOA3 | 20q11 | AIB1 | Nuclear receptor coactivator 3; | 601937 |
| | | | Amplified in breast cancer 1 | |
| NCOA6 | 20q11 | AIB3 | Nuclear receptor coactivator 6; | 605299 |
| | | | Amplified in breast cancer 3 | |
| CYP19 | 15q21.2 | ARO | Aromatase | 107910 |
| STARD3 | 17q12 | MLN64 | Binds & transports cholesterol | 607048 |
| PTGS1 | 9q33.2 | COX1 | Prostaglandin-endoperoxide | 176805 |
| | | | Synthase 1; Cyclooxygenase 1 | |
| PTGS2 | 1q31.1 | COX2 | Prostaglandin-endoperoxide | 600262 |
| | | | Synthase 2; Cyclooxygenase 2 | |
| STS | Xp22.31 | ARSC1 | Steroid sulfatase | 308100 |
| STE | 4q13.1 | EST | Estrogen sulfotransferase | 600043 |
| HSD17B2 | 16q23.3 | HSD17 | 17-beta-hydroxysteroid | 109685 |
| | | | dehydrogenase II | |
| NCOR1 | 17p12-p11.2 | N-CoR | Nuclear receptor | 600849 |
| | | | co-repressor 1 | |
| NCOR2 | 12q24.31 | SMRT | Nuclear receptor | 600848 |
| | | | co-repressor 2 | |
| SAFB | 19p13.3 | HET | Scaffold attachment factor B | 602895 |
| SAFB2 | 19p13.3 | KIAA0138 | Scaffold attachment factor B2 | 608066 |
| BCL2 | 18q21 | Bcl-2 | B-CELL CLL/LYMPHOMA 2 | 151430 |
| SCUB2 | 11p15 | Cegb1, Cegf1 | Signal peptide, CUB and EGF-like | |
| | | | domain-containing protein 2 precursor | |
| FASN | 17q25 | FAS | Fatty acid synthase | |
| BIRC5 | 17q25 | EPR-1 | Inhibitor of apoptosis | 603352 |

In one embodiment, one further step of any of the methods of the invention may comprise determining the status of aberration of one of the ESR1-related gene, e.g. PGR, FASN, COX, SCUBE2, BCL2, BIRC5 or PTGS2. In another embodiment, the further steps may comprise determining the status of aberration of two, three or more ESR1-related genes. It was surprisingly found that determining the status aberration of some panels of ESR1-genes together with determining the status of aberration of the ESR1 gene may be very useful for prognostic purposes of the invention. Non-limited examples of such gene panes are described in EXAMPLES. Thus, in different embodiments the panels comprising 2, 3, 4, 5, 6 or 7 ESR1-related genes may be examined for the presence of aberration.

The status of aberration of ESR1 or an ESR1-related gene is preferably to be determined as the number of copies of the gene in situ. The number of copies of the gene is typically determined as cut-off values. As already mentioned above, in one embodiment, the status of the gene aberration is determined as amplification of the gene sequence in situ or amplification of a part of the gene sequence, which means that the determined status is an increased number copies of the gene sequence in situ. In another embodiment, the aberration may be deletion of the gene, which may be deletion of the whole gene sequence or deletion of parts of the gene sequence, which means that there is a decreased number of copies of or no the gene sequence determined. Still, in another embodiment, the determined status of aberration of the gene may be no aberration, which means that the gene sequence is presented in situ in a normal/usual number of copies.

In one embodiment, the amplified gene sequence, or amplified part of the gene sequence, or amplified sequence of a regulatory element of the gene, e.g. promoter, etc, comprises a mutation which affect the expression of the gene, e.g. leads to a low or no gene expression or to production of non-functional products of the gene, e.g. RNA molecules, proteins.

The status aberration of any of the genes is preferably determined in vitro by in situ hybridisation. A preferable method of in situ hybridization is a Flourescent In Situ Hybridization (FISH) or Chromogen In Situ Hybridization analysis (CISH). However, a combination of different methods of genetic analysis may be used in different embodiments.

According to one embodiment of the invention, the in situ hybridization is performed in vitro using at least one probe targeted at gene region or at a portion of the gene region, e.g. a region of ESR1, and at least one reference probe. Both probes gene targeted and reference probe are preferably selected form the group consisting of nucleic acid, nucleic acid analog and protein probes. Other possible probes for the purposes of the invention are discussed above. In one embodiment, at least one probe which is targeted at gene region is a nucleic acid probe.

In one embodiment, at least one reference probe is targeted at the centromeric region of chromosome, e.g. the centromeric region of chromosome 6 or of any other human chromosome, e.g. chromosomes 1, 2, 11, 17 or 18, as used in the present invention. In one embodiment, the at least one reference probe is a nucleic acid analog probe, e.g. a PNA probe.

In another embodiment, the reference probe may be targeted at a reference sequence located on the opposite arm of a chromosome (opposite to the arm where the target gene sequence is located). It is preferred that such reference sequence is not related to any gene which is aberrant in breast cancer.

The probes are preferably labelled with different labels, such that a label of the gene targeted probe can be distinguished from the label of reference probe, e.g. the labels generate fluorescent light of different wave length or they comprise different enzyme labels or chromophores.

The methods of above relate to therapeutic treatment being either hormonal, non-hormonal chemotherapy or combined. The term “hormonal therapy” in the present context refers to therapeutic treatment comprising using drugs that are targeted at ER such that they modulate expression, metabolism and/or activity of ER in cells of a patient, in particular cancer cells, or has a regulatory effect on gonads or breast tissue. The term “non-hormonal chemotherapy” in the present context refers to therapeutic treatment comprising using drugs which are targeted at other than ER molecules, e.g. cytotoxic chemotherapy and trastuzumab.

Hormonal chemotherapy for breast cancer at present employs (i) selective estrogen-receptor modulators (SERMs), e.g. tamoxifen, raloxifene, faslodex, (ii) aromatase inhibitors, e.g. anastazole, letrozole, exemestane, (iii) ovarian ablation or supressors, e.g. buserlin, goserelin, leuprorelin, nafarelin, (iv) progestins, e.g. medroxyprogesterone acetate and megestrol acetate, (v) estrogens, e.g. estradiol, polyestradiolphosphate, (vi), steroid sulphatase inhibitors, (vii) compounds promoting degradation of ER in cells, e.g. ICI 182,780. The determined status of an ESR aberration is used herein to determine sensitivity of breast cancer lesions to these and similar drugs.

The cancer patient whom the methods relate to is a patient having or suspected of having cancer, wherein cancer may be breast, ovarian, prostate cervical, corpus uteri cancer and endometrial carcinoma.

The status of aberration of any gene of interest is determined in a sample obtained from a cancer patient. It is preferably a tissue sample. The tissue sample may be a biopsy sample, a slice of a frozen tissue section or paraffin embedded tissue section, a sample of smears, exudates, ascites, blood, bone marrow, sputum, urine, or any tissue sample treated with a fixative.

Another aspect of the invention relates to composition comprising comprising at least two probes, e.g. a kit-in-parts wherein at least one probe is for the determining of the status of aberration of ESR1 in situ, and another probe is a reference probe-

Thus, in one embodiment the kit-in-parts comprises at least one probe which is targeted at the ESR1 gene region and at least one probe which is a reference probe. The reference probe is preferably a probe which is targeted at the centromeric region of human chromosome 6 (CEN-6), or a probe which is targeted at the centromeric region of another human chromosome, e.g. chromosome 2 (CEN-2). Preferably, the probe targeted at the ESR1 gene region is a DNA probe and the reference probe is a PNA probe.

In another embodiment the kit-in-parts may comprise several probes targeted at different target genes described above and several reference probes. The reference probes may be probes targeted at centromeric regions of different human chromosomes, preferably, centromeric regions of chromosome 1 (CEN-1), chromosome 2 (CEN-2), chromosome 6 (CEN-6) chromosome 11 (CEN-11), and chromosome 18 (CEN-11). Preferably, the gene targeted probes are DNA probes, and the reference probes are PNA probes. The kit-in-parts of the invention may comprise a combination of any of the gene targeted probes and reference probes. Some combinations of particular target gene and reference probes are shown in Table 2 below.

TABLE 2

| | | | | | | Probe | |
|---|---|---|---|---|---|---|---|
| | | Gene | | Clone | Probe | conc. | |
| Gene | Position | length (kb) | Clone | length (kb) | length (kb) | (ng/μL) | Reference |
| ESR2 | 14q23.2 | 111.1 | CTD-2160J7 | 88.0 | 423.3 | 10.58 | CEN-2 |
| | | | RP11-701L2 | 183.4 | | | |
| | | | CTD-2262M8 | 161.7 | | | |
| PGR | 11q22.1 | 92.1 | RP11-762M1 | 163.3 | 328.3 | 16.41 | CEN-11 |
| | | | CTD-2148J14 | 172.3 | | | |
| SCUBE2 | 11p15.4 | 71.2 | CTD-2541O1 | 179.4 | 341.0 | 17.05 | CEN-11 |
| | | | RP11-64B2 | 161.5 | | | |
| BCL2 | 18q21.33 | 195.4 | RP11-299P2 | 146.7 | 358.1 | 13.43 | CEN-18 |
| | | | RP11-111L3 | 165.7 | | | |
| | | | RP11-876G20 | 177.4 | | | |
| BIRC5 | 17q25.3 | 11.4 | CTD-2523I20 | 156.7 | 329.7 | 9.26 | CEN-17 |
| | | | CTD-3234C5 | 203.5 | | | |
| PTGS2 | 1q31.1 | 8.6 | RP11-338J18 | 153.7 | 329.2 | 5.74 | Cent 1 |
| | | | RP11-348G9 | 178.8 | | | |
| FASN | 17q25.3 | 19.9 | CTD-3118M1 | 149.4 | 370.4 | 9.26 | CEN-17 |
| | | | RP11-1087N2 | 220.8 | | | |

As mentioned above, each probe of the kit may comprises a label. The label of the probe targeted at the gene region is preferably different form the label of the reference probe.

The labels may be selected from fluorescent, chromogen or enzyme labels. Preferably, the label of the probe which is targeted at a target gene region and the label of the probe which is targeted at a centromeric region are two different fluorescent labels. In another preferred embodiment, the labels are two different chromogen labels. In another preferred embodiment, the labels are two different enzyme labels.

Analysis of samples using in situ hybridization and evaluation of the results may be performed by using manual or partially or fully automated protocols.

In one embodiment of the invention, the method further utilizes image analysis systems.

Manual reading of the result of many samples is very time consuming. Therefore, it would be a great help to have access to automated systems. The reading of, for example, many fields of hybridization would be aided by fluorescence image analysis with high speed scanning facilities. MetaSystems is an example of a provider of an image analysis system that might be used.

All the above embodiments are illustrated below by not-limited examples below.

### EXAMPLES

### Example 1

### Probes

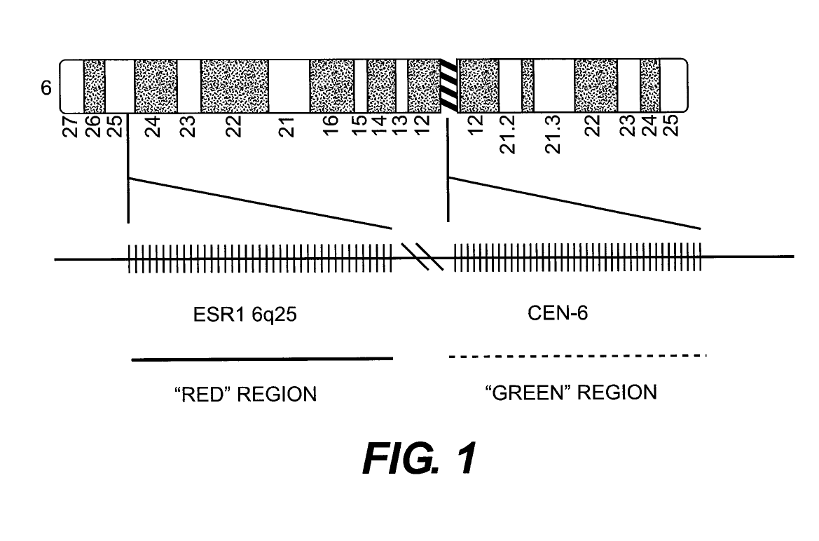

FIG. 1 demonstrates the general design of the FISH probe mix for detection of ESR1 gene copy number used in the experiments described in the below examples. The probe is constructed as a mixture of Texas Red and Fluorescein labeled probes in which the red BAC (Bacterial Artificial Chromosome) DNA based probe is specific for the ESR1 gene at 6q25 and the green PNA (Peptide Nucleic Acid) based reference probe is specific for the centromeric region of chromosome 6.

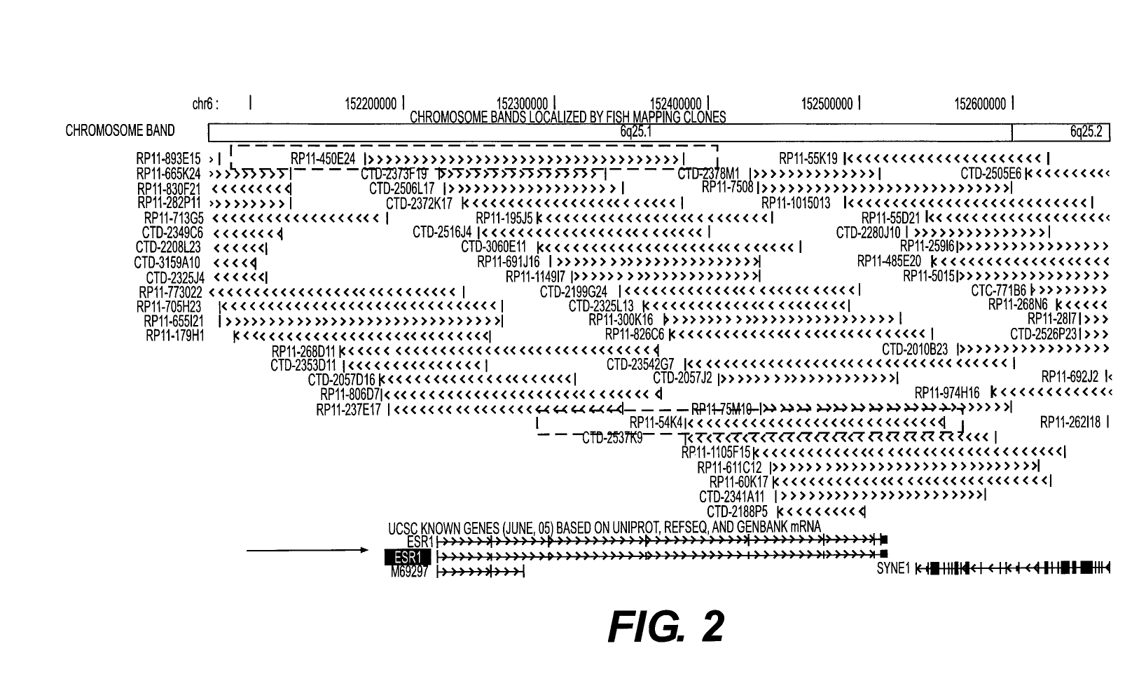

FIG. 2 demonstrates the positions at chromosome 6q25 of the BAC clones used for construction of the ESR1 probe (marked in rectangles), relative to the position of the genomic ESR1 sequence (marked with an arrow).

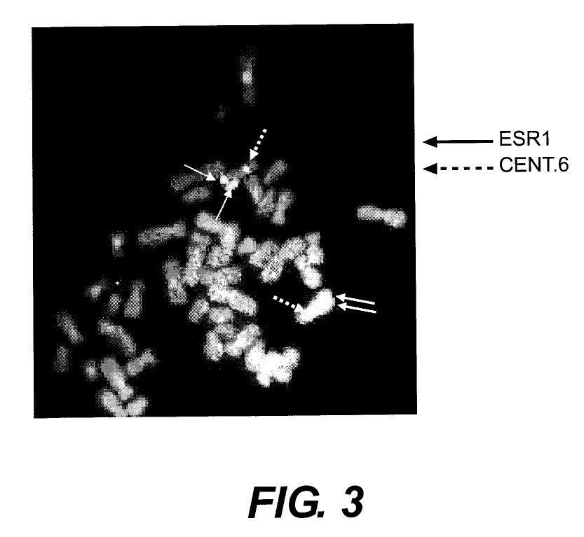

The ESR1 genomic sequence is located on the chromosome 6 q-arm, region 2 band 5 (6q25) where it covers 295.721 bp from position 152.220.800 to 152.516.520. The source of the labeled DNA probe is the two BAC clones RP11-450E24 and RP11-54K4, together covering position 152.175.459 to 152.555.252 (except for a 166 bp gab between the two BAC clone inserts). Identity verification of the BAC clones used for the ESR1 probe has been performed by restriction analysis, BAC end sequencing and in situ hybridization of the purified Texas Red labeled BAC DNA to normal human blood metaphase samples (FIG. 3).

The chromosome 6 reference probe is composed of a mixture of fluorescein labelled PNA oligo constructs complementary to α-satellite repeat sequences specific for the chromosome 6 centromeric region. The below examined mixture is composed of four different PNA oligos. The individual PNA oligos were designed, synthesized and selected by functional examination by Dako Denmark A/S and combined in a CEN-6 specific mixture.

FIG. 3 demonstrates the specific hybridization of the ESR1 probe mixture to 6q25 (four red signals—indicated by 4 solid arrows) and the centromeric region of chromosome 6 (two green signals—indicated by 2 dotted arrows) to normal human metaphase spreads.

The following chart presents a summary of probes of other genes used in the experiments described in the examples below:

ESR2

| | |
|---|---|
| Gene position 14q23.2 | 61,200,001-64,000,000 |
| Length of ESR2 | 63,763,506-63,874,563→ 111,058 bp |
| Clones: | |
| CTD-2160J7*(u) | 63,685,869-63,773,843 → 87,975 bp |
| Blast reference gene(s): SYNE2 | |
| RP11-701L2*(m) | 63,768,483-63,951,855→ 183,373 bp |
| Blast reference gene(s): ESR2 | |
| CTD-2262M8(d) | 63,947,505-64,109,203→ 161,699 bp |
| Blast reference gene(s): MTHFD1 and AKAP5 | |
| Combined probe length | 64,109,203-63,685,869 = 423,334 bp |

PGR

| | |
|---|---|
| Gene position 11q22.1 | 96,400,001-101,200,000 |
| Length of PGR | 100,414,313-100,506,465 → 92,153 bp |
| Clones: | |
| RP11-762M1*(u) | 100,411,738-100,575,015 → 163,278 bp |
| Blast reference gene(s): PGR | |
| CTD-2148J14(d) | 100,567,771-100,740,030 → 172,260 bp |
| Blast reference gene(s): PGR and TRPC6 | |
| Combined probe length | 100,740,030-100,411,738 = 329,222 bp |

SCUBE2

| | |
|---|---|
| Gene position 11p15.4 | 2,800,001-10,700,000 |
| Length of SCUBE2 | 8,998,511-9,069,731→ 71,221 bp |
| Clones: | |
| CTD-2541O1(u) | 8,805,269-8,984,697→ 179,429 bp |
| Blast reference gene(s): c11orf17 | |
| RP11-64B2*(d) | 8,984,725-9,146,246→ 161,522 bp |
| Blast reference gene(s): SCUBE2 and RAB6IP1 | |
| Combined probe length | 9,146,246-8,805,269 = 340,977 bp |

BCL2

| | |
|---|---|
| Gene position 18q21.33 | 57,100,001-59,800,000 |
| Length of BCL2 | 58,941,559-59,136,910 → 195,352 bp |
| Clones: | |
| RP11-299P2*(u) | 58,927,479-59,074,166 → 146,688 bp |
| Blast reference gene(s): BCL2 alpha isoform | |
| RP11-111L3*(m) | 58,986,452-59,152,195 → 165,744 bp |
| Blast reference gene(s): BCL2 alpha and beta isoform | |
| RP11-876G20*(d) | 59,108,201-59,285,610 → 177,410 bp |
| Blast reference gene(s): BCL2 alpha and beta isoform | |
| Combined probe length | 59,285,610-58,927,479 = 358,131 bp |

BIRC5

| | |
|---|---|
| Gene position 17q25.3 | 72,900,001-78,774,742 |
| Length of BIRC5 | 73,721,872-73,733,310→ 11,439 bp |
| Clones: | |
| CTD-2523I20*(u) | 73,693,063-73,849,793→ 156,731 bp |
| Blast reference gene(s): TK1 and AFMID | |
| CTD-3234C5(d) | 73,819,236-74,022,743 → 203,508 bp |
| Blast reference gene(s): SOCS3 and PGS1 | |
| Combined probe length | 74,022,743-73,693,063 = 329,680 bp |

FASN

| | |
|---|---|
| Gene position 17q25.3 | 72,900,001-78,774,742 |
| Length of FASN | 77,629,504-77,649,395→ 19,892 bp |
| Clones: | |
| CTD-3118M1*(u) | 77,573,012-77,722,456 → 149,445 bp |
| Blast reference gene(s): STRA13 and LRRC45 | |
| RP11-1087N2(d) | 77,722,590-77,943,417→ 220,828 bp |
| Blast reference gene(s): CCDC57 and SLC16A3 | |
| Combined probe length | 77,573,012-77,943,417 = 370,405 bp |

(u)—upstream
(m)—middle
(d)—downstream
*clone covering gene region

### Protocols

Protocol 1: Verification of BAC clones: Each BAC clone was streaked on Luria-Bertani (LB), chloramphenicol agar plates (3% LB-Broth agar, 2% glucose, 20 μg/mL chloramphenicol) and incubated at 37° C. overnight. Pre-cultures consisting of a single, isolated colony inoculated in 10 mL LB, chloramphenicol liquid medium (2.5% LB-Broth base medium, 10 mM Tris-HCl pH 7.5, 20 μg/mL chloramphenicol) were incubated overnight at 37° C. at vigorous stirring (200-250 rounds per minute (rpm)) to ensure good aeration. Glycerol-stocks (20%) for long term storage at −70° C. were prepared and the rest of the bacteria were used for DNA fragmentation. The introductory steps from stab culture to liquid pre-culture were repeated with BAC clones from one of the glycerol stocks and new glycerol stocks were made. Finally, clones from the latter glycerol stocks were again streaked out on LB, chloramphenicol agar plates and incubated overnight at 37° C. Subsequently, five isolated colonies were inoculated separately in 10 mL LB, chloramphenicol liquid medium and incubated at 37° C. overnight at stirring (200-250 rpm). The five clones were analyzed by DNA fragmentation with BamHI.

Protocol 2: Purification: The cultures for restriction enzyme analysis were purified using the QIAGEN Plasmid MAXI kit. The bacteria were harvested by centrifugation in a Beckman centrifuge at 4,000 g for 10 min. The bacterial pellet was resuspended on ice in 0.4 mL cold P1 resuspension buffer containing RNase A (100 μg/mL). 0.4 mL P2 lysis buffer (SDS, NaOH) was added, mixed by inverting the tube 6 times, and incubated for 5 min at room temperature. Hereafter, 0.4 mL cold P3 neutralizing buffer (potassium acetate) was added and the tube was again inverted 6 times and incubated 10 min on ice. The tube was centrifuged in an Ole Dich Microcentrifuge at 4° C., 20,000 g for 15 min. The supernatant containing plasmid DNA was subsequently collected. 0.7 mL isopropanol was added and the contents were centrifuged at 4° C., 20,000 g for 30 min in an Ole Dich Microcentrifuge. The supernatant was discarded and the pellet was washed with 0.5 mL 70% EtOH. Without resuspension the tube was centrifuged in an Ole Dich 4° C., 20,000 g in 5 min. The supernatant was removed and the pellet air dried for 10-20 min and afterwards resuspended in 25 μL TE buffer (10 mM Tris-HCl, 0.1 mM EDTA, pH 8.0).

Protocol 3: DNA Fragmentation: 17 μL DNA solution was used for the BamHI DNA fragmentation. The plasmid DNA was kept on ice and mixed with 2 μL 10× concentrated REact^{R}3 reaction buffer. 1 μL BamHI (10 U/μL) was added and the mixture was incubated at 37° C. for 2 hours. Following incubation, the mixtures were placed on ice and 2.2 μL 10× gel loading buffer added. DNA fragments were separated by gel electrophoresis using 0.8% agarose (Seakem Gold) gel in 1×TAE buffer (40 mM Tris-HCL, 0.1 mM EDTA) supplemented with ethidium bromide (0.4 μg/mL). 10 μL 1 Kb Plus DNA ladder was used as reference. The gel was run at 30V for about 16 hours. Following electrophoresis the gel was placed under UV light and a digital photo was taken.

Protocol 4: Propagation Protocol: From one of the last produced glycerol stocks performed in the verification process (protocol 1), bacterial solution was streaked on LB, chloramphenicol agar plates (3% LB-Broth agar, 2% glucose, 20 μg/mL chloramphenicol) and incubated at 37° C. overnight. A pre-culture was performed by inoculating a single, well-isolated colony in 25 mL LB chloramphenicol liquid medium (2.5% LB-Broth base, 10 mM Tris-HCl, pH 7.5, 20 μg/mL chloramphenicol). The pre-culture was incubated at 37° C. overnight at vigorous stirring (200-250 rpm). The pre-culture was inoculated into 1 L pre-heated LB, chloramphenicol liquid medium and incubated for 5 hours at stirring (200-250 rpm) at 37° C. At 0, 2.5, and 5 hours, the optical density at 600 nm (OD₆₀₀) was measured. After 5 hours, the bacteria were harvested using a Beckman centrifuge (JA 10) 6,000 rpm for 15 min at 4° C. The supernatant was removed and the DNA was subsequently purified from the pellet, see protocol 5.

Protocol 5: Purification of plasmid DNA after propagation: The Macherey-Nagel Nucleobond® Xtra Kit was used to purify large scale BAC DNA. After harvesting, the bacterial pellet from the 1 L culture was resuspended in 60 mL cold Nucleobond® Xtra RES buffer solution containing RNase A (100 μg/mL), kept on ice. The bacteria were lysed by adding 60 mL Nucleobond® Xtra LYS buffer solution (NaOH, SDS). The tube was inverted 6 times and incubated 5 min at room temperature. 60 mL cold Nucleobond® Xtra NEU buffer solution (potassium acetate) was subsequently added, the tube was again inverted 6 times, and incubated 10 min on ice. The solution was centrifuged (Beckman JA-10) at 9,500 rpm for 30 min at 4° C. The Nucleobond® Xtra Maxi Column and filter were prepared by adding 25 mL Nucleobond® Xtra EQU buffer solution. The supernatant containing plasmid DNA was added to the column. 15 mL Nucleobond® Xtra EQU solution buffer was added when the supernatant had run through and the filter was removed afterwards. 25 mL Nucleobond® Xtra WASH buffer solution was added and the BAC DNA was eluted by 15 mL Nucleobond® Xtra ELU buffer solution. Afterwards, the plasmid DNA was precipitated with 10.5 mL 0.7 volume isopropanol and the solution was centrifuged (Beckman JA-20) at 14,000 rpm for 30 min at 4° C. The supernatant was removed and the DNA pellet was washed by adding 5 mL room-temperature 70% ethanol and followed by centrifugation at 14,000 rpm for 10 min at 4° C. The tube containing the DNA pellet was air-dried for approximately 20 min. Afterwards, the DNA pellet was dissolved in 500 μL TE-buffer (10 mM Tris-HCl, 0.1 mM EDTA, pH 8.0) and stored at <−18° C.

Protocol 6: DNA Fragmentation: Purified DNA was characterized by DNA fragmentation using the enzymes BamHI and KpnI. 2 μg DNA was diluted in sterilized MilliQ water to a volume of 17 μL. The DNA solutions were mixed with 2 μL 10× concentrated REact^{R}3 and REact^{R}4 restriction buffers for BamHI and KpnI, respectively. 1 μL restriction enzyme was added and the mixtures were incubated at 37° C. for 2 hours. Following incubation the mixtures were placed on ice and 2.2 μL 10× gel loading buffer added. DNA fragments were separated by gel electrophoresis using a 0.8% agarose (Seakem Gold) gel in 1×TAE buffer (40 mM Tris-HCL, 0.1 mM EDTA) supplemented with ethidium bromide (0.4 μg/mL). 10 μL 1 Kb Plus DNA ladder was used as reference. The gel was run at 30V for approximately 16 hours. Following electrophoresis, the gel was placed under UV light and a digital photo was taken.

Protocol 7: Texas Red Nick Translation Labeling: All samples and reagents were kept on ice. 15 μg of purified DNA was used for each Nick Translation. DNA was mixed with sterilized MilliQ water in an eppendorf tube to a total volume of 180 μL. 60 μL 5× Fluorophore labeling mix (2.5 mM dATP, 2.5 mM dGTP, 2.5 mM dTTP, 2.5 mM dCTP, 1.0 mM Texas Red-X-OBEA-dCTP in Sterilized MilliQ water) was added and the mixture vortexed and centrifuged before adding 60 μL Nick Translation Mix (DNase I and E. coli DNA polymerase I). The solution was gently mixed and centrifuged before 6 hours of incubation in water bath at 15° C. The reaction was inactivated by adding 15 μL EDTA (0.5M) and incubating the mixture at 65° C. in water bath for 10 min to denature the enzymes. The mixture was afterwards placed on ice.

Protocol 8: Purification of Labeled Probes: Purification was performed on NICK Sephadex G-50 columns. After labeling, the mixtures were freeze-dried at Speed Vac. The mixtures were re-dissolved in 30 μL sterilized MilliQ water. The column was emptied, prepared by washing with 3 mL TE-buffer (1 mM Tris-HCl, 10 μM EDTA, pH 8.0), and subsequently equilibrated with 3 ml TE-buffer. When TE-buffer had run through, the labeled probe solution was added. 400 μL TE-buffer was added and the run-through was discarded. The labeled DNA was eluted with an additional 400 μL TE-buffer. The run-through was evaporate at Speed Vac to obtain a sample volume of approximately 100-150 μL (˜250 ng/μL).

Protocol 9: Agarose Gel Electrophoresis of Labeled Probes: The labeled DNA fragments were separated by agarose gel electrophoresis. 500 ng labeled DNA was diluted with MilliQ water to a total volume of 20 μL. The DNA mixture was denatured at 95° C. for 3 min and placed on ice. The DNA was separated at a 2% agarose gel with an E-gel system with a 50 bp DNA ladder as reference. The gel was left running for around 30 min at 60 V and until the marker was 2 cm from the bottom of the gel. Following electrophoresis the gel was placed under UV light and a digital photo was taken.

### Example 2

### ESR1 Gene Copy Numbers in Non-Malignant Breast Samples

Patient samples: Samples from 120 patients having surgery to reduce the size of the breasts were collected at Herlev University Hospital. The tissue blocks were collected from the archives of the Department of Pathology, and were investigated with H&E staining to ensure that the tissue was non-cancerous. 6 tissue micro arrays (TMAs) were produced with 2.0 μm cores from each patient. Each TMA contained samples from 20 patents along with 2 control samples.

FISH analysis: The FISH assays were performed according to Protocols 10 or 11 (below).

Protocol 10: Cytology FISH: The slides were pre-treated for 2 min in 3.7% formaldehyde (pH 7.6) at room temperature. The slides were washed in 1× Wash Buffer for 2×5 min at room temperature. Afterwards, the target tissue was dehydrated in a cold series of EtOH (70%, 85% and 96%) 2 min each and air-dried. On each target area, 10 μL hybridization mixture (target and reference probe diluted in hybridization buffer: 45% formamid, 10% dextran sulphate, 0.3M NaCl, 5 mM sodium phosphate and PNA blocking sequences) was added. Coverslips were applied to cover the hybridization area and the edges of the coverslips were sealed with rubber cement. The slides were placed in a Hybridizer™, denatured at 82° C. for 5 min and subsequently, hybridized at 45° C. for 14-20 hours. After hybridization the coverslips were removed and the slides were placed in 1× Stringency Wash Buffer at room temperature and afterwards rinsed in 1× Stringency Wash Buffer preheated to 65° C. for 10 min. The slides were washed 2×3 min in 1× Wash Buffer at room temperature. Next, the tissue was dehydrated in a series of cold EtOH increasing in strength, 70%, 85% and 96%, 2 min in each and air-dried. Each slide was mounted with 15 μL mounting-medium anti-fade solution with DAPI, sealed with coverslips, and stored in the dark before signal detection.

Protocol 11: Histology FISH: Paraffin from the tumor material was removed by placing the slides in xylene in 2×5 min. The tissue was subsequently rehydrated in 2×2 min in 96% EtOH and 2×2 min in 70% EtOH. The slides were washed 2 min in 1× Wash Buffer at room temperature. Slides were immersed 10 min in 1× Pretreatment solution. The solution containing the slides was pre-heated and then incubated at 100° C. in 10 min using a microwave. The slides were allowed to cool in the pretreatment solution for 15 min. Subsequently, the slides were washed 2×2 min in 1× Wash Buffer at room temperature. Surplus solution was removed before 5-8 drops of cold Ready-to-use-Pepsin was added at the target area of each slide before incubation for 3 min at 37° C. Afterwards, the slides were washed 2×3 min in Wash Buffer and dehydrated in a series of cold EtOH, 2 min in 70%, 85%, and 96%, respectively. The slides were air-dried and 10-15 μL of probe mixture was added (target probe, reference probe, and hybridization buffer: 45% formamid, 10% dextran sulphate, 0.3M NaCl, 5 mM sodium phosphate and PNA blocking sequences) for separate tissue samples and 25 μL for TMAs. The slides were covered with coverslips and sealed with rubber cement, incubated in a Hybridizer™, 5 min at 82° C., and subsequently at 45° C. for 14-20 hours. After hybridization, glue and coverslips were removed and the slides were placed in 1× Stringent Wash Buffer at room temperature before washing the slides for 10 min in 1× Stringent Wash Buffer, preheated to 65° C. Slides were washed in 2×3 min in 1× Wash Buffer at room temperature, and dehydrated in a series of cold EtOH solutions 2 min in 70%, 85%, and 96%, respectively. The slides were air-dried for approximately 20 min and counterstained with 15 μl of Fluorescence Mounting Medium (DAPI and antifade) for single tissue samples and 25 μL for TMAs. The slides were mounted and stored in the dark before signal enumeration.

The ratio between red and green signals was evaluated by FISH on 120 patient samples containing normal cells. A total of 60 cells were counted per patient, and only cells containing both one red and one green signal were evaluated. Signals of the same color with a distance less than or equal to the diameter of the signals were evaluated as one.

Results:

Each nuclei of a normal, non-cancerous cell may contain 2 red signals from the ESR1 probe and 2 green signals from the CEN-6 reference probe. However, due to the fact that nuclei present in paraffin-embedded tissue are often larger than the thickness of the cut-section, it is important to establish a reference interval that clearly discriminates between cancer tissue and normal, non-cancerous tissue.

From each sample, 60 nuclei were scored, and the numbers of red and green signals were counted. The number of red signals varied between 95-115 signals in 60 nuclei with an average of 1.78 red signals per cell. The number of green signals varied between 84-112 signals in 60 nuclei with and average of 1.69 green signals per cell. The ratio for each sample varied from 0.96 to 1.29 with an average of 1.06±0.04.

Two samples were considered as outliers. They appeared abnormal with too many red signals. These 2 samples were replaced by 2 other samples in the cohort as they could not be scored initially. The 2 outliers were stained again and rescored successfully. One sample showed a ratio of 1.40 while the other had a ratio of 2.17. According to the guidelines for HER2 scoring, a ratio between 1.8 and 2.2 is considered borderline and should be rescored (Wolff et al, American Society of Clinical Oncology/College of American Pathologists guideline recommendations for human epidermal growth factor receptor 2 testing in breast cancer. J Clin Oncol. 2007, 25(1):118-45),

Table 3 (below) shows the results of the analysis of the 120 patient samples

TABLE 3

| | No. | | | |
|---|---|---|---|---|
| | counted | ESR1 | Ref. | |
| Nr. | cells | count | Count | Ratio |
| 1 | 60 | 108 | 100 | 1.08 |
| 2 | 60 | 107 | 109 | 0.98 |
| 3 | 60 | 107 | 104 | 1.03 |
| 4 | 60 | 113 | 112 | 1.01 |
| 5 | 60 | 110 | 100 | 1.10 |
| 6 | 60 | 107 | 110 | 1.0 |
| 7 | 60 | 103 | 98 | 1.05 |
| 8 | 60 | 111 | 98 | 1.1 |
| 9 | 60 | 105 | 103 | 1.02 |
| 10 | 60 | 110 | 98 | 1.12 |
| 11 | 60 | 115 | 107 | 1.07 |
| 12 | 60 | 107 | 105 | 1.02 |
| 13 | 60 | 106 | 100 | 1.06 |
| 14 | 60 | 110 | 101 | 1.09 |
| 15 | 60 | 112 | 98 | 1.14 |
| 16 | 60 | 114 | 111 | 1.03 |
| 17 | 60 | 113 | 109 | 1.04 |
| 18 | 60 | 104 | 96 | 1.08 |
| 19 | 60 | 101 | 97 | 1.04 |
| 20 | 60 | 107 | 107 | 1.00 |
| 21 | 60 | 103 | 97 | 1.06 |
| 22 | 60 | 103 | 96 | 1.07 |
| 23 | 60 | 105 | 99 | 1.06 |
| 24 | 60 | 106 | 104 | 1.02 |
| 25 | 60 | 108 | 101 | 1.07 |
| 26 | 60 | 102 | 99 | 1.03 |
| 27 | 60 | 106 | 103 | 1.03 |
| 28 | 60 | 103 | 97 | 1.06 |
| 29 | 60 | 99 | 95 | 1.04 |
| 30 | 60 | 105 | 95 | 1.11 |
| 31 | 60 | 105 | 99 | 1.06 |
| 32 | 60 | 98 | 95 | 1.03 |
| 33 | 60 | 96 | 93 | 1.03 |
| 34 | 60 | 106 | 94 | 1.13 |
| 35 | 60 | 103 | 102 | 1.01 |
| 36 | 60 | 108 | 101 | 1.07 |
| 37 | 60 | 95 | 96 | 0.99 |
| 38 | 60 | 103 | 102 | 1.01 |
| 39 | 60 | 107 | 95 | 1.13 |
| 40 | 60 | 111 | 101 | 1.10 |
| 41 | 60 | 110 | 105 | 1.05 |
| 42 | 60 | 110 | 104 | 1.06 |
| 43 | 60 | 100 | 100 | 1.00 |
| 44 | 60 | 109 | 105 | 1.04 |
| 45 | 60 | 101 | 97 | 1.0 |
| 46 | 60 | 110 | 108 | 1.02 |
| 47 | 60 | 107 | 99 | 1.08 |
| 48 | 60 | 102 | 102 | 1.00 |
| 49 | 60 | 103 | 98 | 1.05 |
| 50 | 60 | 99 | 92 | 1.08 |
| 51 | 60 | 107 | 96 | 1.11 |
| 52 | 60 | 102 | 101 | 1.01 |
| 53 | 60 | 108 | 102 | 1.06 |
| 54 | 60 | 113 | 108 | 1.05 |
| 55 | 60 | 108 | 97 | 1.11 |
| 56 | 60 | 107 | 104 | 1.03 |
| 57 | 60 | 105 | 95 | 1.11 |
| 58 | 60 | 110 | 109 | 1.01 |
| 59 | 60 | 104 | 94 | 1.11 |
| 60 | 60 | 108 | 84 | 1.29 |
| 61 | 60 | 112 | 112 | 1.00 |
| 62 | 60 | 108 | 102 | 1.06 |
| 63 | 60 | 98 | 96 | 1.02 |
| 64 | 60 | 106 | 101 | 1.05 |
| 65 | 60 | 113 | 111 | 1.02 |
| 66 | 60 | 100 | 97 | 1.03 |
| 67 | 60 | 105 | 109 | 0.96 |
| 68 | 60 | 105 | 101 | 1.04 |
| 69 | 60 | 113 | 104 | 1.09 |
| 70 | 60 | 106 | 106 | 1.00 |
| 71 | 60 | 107 | 104 | 1.03 |
| 72 | 60 | 108 | 101 | 1.07 |
| 73 | 60 | 113 | 104 | 1.09 |
| 74 | 60 | 111 | 106 | 1.05 |
| 75 | 60 | 108 | 101 | 1.07 |
| 76 | 60 | 109 | 104 | 1.05 |
| 77 | 60 | 110 | 107 | 1.03 |
| 78 | 60 | 103 | 97 | 1.06 |
| 79 | 60 | 108 | 103 | 1.05 |
| 80 | 60 | 108 | 99 | 1.09 |
| 81 | 60 | 101 | 96 | 1.05 |
| 82 | 60 | 112 | 105 | 1.07 |
| 83 | 60 | 113 | 110 | 1.03 |
| 84 | 60 | 98 | 94 | 1.04 |
| 85 | 60 | 106 | 105 | 1.01 |
| 86 | 60 | 111 | 107 | 1.04 |
| 87 | 60 | 112 | 102 | 1.10 |
| 88 | 60 | 107 | 99 | 1.08 |
| 89 | 60 | 103 | 96 | 1.07 |
| 90 | 60 | 107 | 105 | 1.02 |
| 91 | 60 | 105 | 97 | 1.08 |
| 92 | 60 | 103 | 100 | 1.03 |
| 93 | 60 | 109 | 111 | 0.98 |
| 94 | 60 | 110 | 105 | 1.05 |
| 95 | 60 | 111 | 102 | 1.09 |
| 96 | 60 | 113 | 99 | 1.14 |
| 97 | 60 | 110 | 106 | 1.04 |
| 98 | 60 | 106 | 101 | 1.05 |
| 99 | 60 | 104 | 99 | 1.05 |
| 100 | 60 | 106 | 103 | 1.03 |
| 101 | 60 | 108 | 98 | 1.10 |
| 102 | 60 | 115 | 106 | 1.08 |
| 103 | 60 | 112 | 106 | 1.06 |
| 104 | 60 | 106 | 97 | 1.09 |
| 105 | 60 | 110 | 101 | 1.09 |
| 106 | 60 | 105 | 96 | 1.09 |
| 107 | 60 | 109 | 108 | 1.01 |
| 108 | 60 | 109 | 103 | 1.06 |
| 109 | 60 | 110 | 101 | 1.09 |
| 110 | 60 | 110 | 103 | 1.07 |
| 111 | 60 | 110 | 102 | 1.08 |
| 112 | 60 | 107 | 101 | 1.06 |
| 113 | 60 | 107 | 99 | 1.08 |
| 114 | 60 | 113 | 104 | 1.09 |
| 115 | 60 | 115 | 106 | 1.08 |
| 116 | 60 | 109 | 101 | 1.08 |
| 117 | 60 | 106 | 94 | 1.13 |
| 118 | 60 | 103 | 98 | 1.05 |
| 119 | 60 | 111 | 102 | 1.09 |
| 120 | 60 | 108 | 98 | 1.10 |

Discussion and Conclusion: The data shows that normal breast specimens, when analyzed for ESR1 copy numbers, yield abnormal results in only one of 122 specimens. One additional case was considered an outlier; however, rescoring showed a normal, although elevated ratio of 1.4.

The remaining 120 cases show an average ratio of 1.06 with a standard deviation of 0.04. Using a range of 3 standard deviations (99% interval) the range for normal ratios are 0.93-1.19. According to this interval, additionally one case with a ratio of 1.29 should have been classified as outlier. In addition to the established cut-off for HER2 of 0.8-2.0, alternative ranges for normal ratios can be considered: 0.93-1.19, 0.9-1.3 or 0.85-1.5.

The reason for the calculated average ratio is that the theoretical value of 1.0 may be connected with the fact that the green reference signals originate from a centromeric probe. Centromeric sequences frequently adhere to the nuclei membrane and because of that, cutting the tissue sections leads to the loss of more green signals than red. Theoretically, a normal cell should have a ratio of 1.0, but the actual value is 1.06. By analogy, a tetraploid cell with loss of 1 gene copy will have a ratio 0.75 (3/4), but adding 6% will give an actual value of 0.8. A triploid cell with gain of 1 gene copy will have a theoretical ratio of 1.5, and adding 6% will give an actual ratio of 1.6. Therefore, the range for normal samples could be 0.8-1.6 instead of 0.8-2.0. In Examples 2 to 4 presented herein, the established cut-off values from HER2 guidelines i.e. 0.8-2.0, have been followed.

### Example 3

### Detection of ESR1 Gene

The initial experiment which demonstrated the existence of ESR1 deletions was made on nine FFPE mamma carcinoma tissues identified as ER negative by IHC testing. The nine tissues were taken from the Dako tissue bank; there is no further information on the tissue samples. The nine tissues were hybridized with the ESR1/CEN-6 FISH Probe Mix by use of standard methods and reagents (Dako Histology FISH Accessory Kit K5599).

The hybridized samples were scored by two technicians, each counting at least 60 red signals, with the results as shown in Table 4:

TABLE 4

| | Ratio | Ratio |
|---|---|---|
| | identified | identified |
| Tissue ID | by DBR | by ANA |
| 73/97 50990 | 0.73 | 0.66 |
| 124/97 51037 | 0.77 | 0.92 |
| 74/97 50991 | 0.85 | 0.72 |
| 75/97 50992 | 0.68 | 0.54 |
| 88/97 51001 | 0.55 | 0.77 |
| 123/97 51036 | 0.86 | 0.91 |
| 33/97 50906 | 0.67 | — |
| 34/97H 50908 | 0.91 | 0.96 |
| 57/97 50941 | 0.73 | 0.79 |

When deletions are defined according to the current standard (ratio≦0.8), the two technicians identified 6 and 5, respectively, of the 9 samples as deleted cases with four cases identified as deleted by both technicians. Irrespective of the interpersonal variation, the experiment clearly pointed at ESR1 deletions as a common phenomenon in ER negative mamma carcinoma samples. As ESR1 deletions never have been reported before, a larger study was initiated as described in EXAMPLE 5

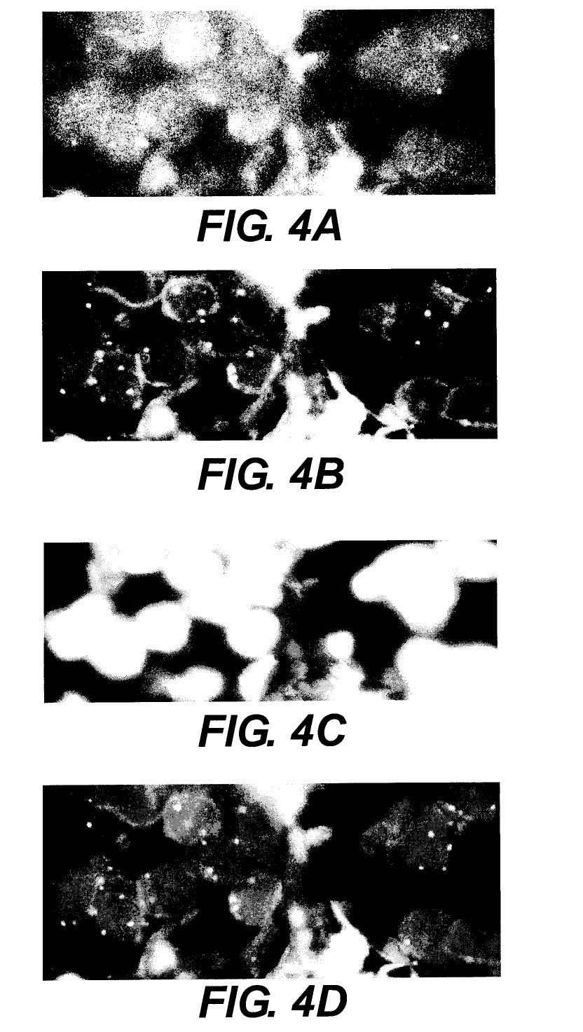

FIG. 4 shows the ESR1/CEN-6 FISH Probe Mix on a mamma carcinoma FFPE tissue with ESR1 deletion. A=ESR1 probe (Texas Red filter); B=CEN-6 probe (Fluorescein filter); C=DAPI counterstain (DAPI filter); D=ESR1/CEN-6 FISH Probe Mix in triple filter

### Example 4

### ESR1 Gene Copy Numbers in Samples from Patients Treated with Tamoxifen

The estrogen receptor (ER) is the target of tamoxifen, and patients with ER negative breast cancer are unlikely to benefit from tamoxifen. Unfortunately, endocrine therapies do not benefit all patients with ER positive tumors and we therefore speculated that copy number changes in the ESR1 gene, coding for the estrogen receptor, confer resistance.

Patient samples: Within a consecutive series of postmenopausal patients allocated to tamoxifen 20 mg daily for 5 years following radical surgery for early breast cancer, we identified 61 patients with recurrence less than 4 years and 48 patients with recurrence more than 7 years after initiation of adjuvant tamoxifen. Archival tissue from the primary tumor was available from 100 of the 109 patients (92%). Samples from 100 breast cancer patients were collected at 4 departments of pathology (University Hospital of Herlev, Roskilde, Glostrup and Gentofte). The tissue blocks were collected from the archives of the Department of Pathology, and cut sections were analyzed.

FISH analysis: The tumor samples were analyzed for ESR1 copy number changes using FISH. The FISH assay was performed according to protocols described in Example 2. The ratio between red and green signals was evaluated by FISH on 100 patient samples. A total of 60 cells were counted per patient, and only cells containing both one red and one green signal were evaluated. Signals of the same color with a distance less than or equal to the diameter of the signals were evaluated as one.

Results: The FISH analysis for ESR1 was successful in 94 of the 100 patients (94%). Amplification was observed in 11 of 52 (21%) with an early recurrence (<4 years), compared to 2 of 42 (5%) patients still recurrence free after more than 7 years (p=0.03).

Table 5 below shows the distribution of aberrations in the 2 patient groups and Table 6 gives the scoring details for all patients.

TABLE 5

| | Relapse < 3 years | Relapse > 7 år | Total |
|---|---|---|---|
| ESR1 Normal | 41 | 40 | 81 |
| ESR1 amplification | 11 | 2 | 13 |
| Total | 52 | 42 | 94 |

P = 0.033

TABLE 6

| Patients with relapse | | | | | Patients without relapse | | | | |
|---|---|---|---|---|---|---|---|---|---|
| within 3 years | | | | | more than 7 years | | | | |
| | No. counted | ESR1 | Ref. | | | No. counted | ESR1 | Ref. | |
| Study no. | cells | score | Score | Ratio | Study no. | cells | score | Score | Ratio |
| 1 | 30 | 65 | 66 | 0.98 | 37 | N/S | N/S | N/S | |
| 2 | 60 | 61 | 65 | 0.94 | 38 | 32 | 72 | 60 | 1.20 |
| 3 | 40 | 70 | 66 | 1.06 | 39 | 27 | 84 | 52 | 1.62 |
| 4 | 14 | 78 | 26 | 3.00 | 40 | 20 | 76 | 62 | 1.23 |
| 5 | 12 | 72 | 23 | 3.13 | 41 | 30 | 71 | 50 | 1.42 |
| 6 | N/S | N/S | N/S | | 42 | 48 | 194 | 77 | 2.52 |
| 7 | 40 | 79 | 71 | 1.11 | 43 | 24 | 60 | 44 | 1.36 |
| 8 | 32 | 65 | 58 | 1.12 | 44 | 28 | 71 | 57 | 1.25 |
| 9 | 12 | 68 | 26 | 2.62 | 45 | 32 | 94 | 55 | 1.71 |
| 11 | 30 | 74 | 54 | 1.37 | 54 | 35 | 67 | 62 | 1.08 |
| 12 | 23 | 62 | 51 | 1.22 | 55 | 27 | 72 | 64 | 1.13 |
| 13 | 27 | 70 | 42 | 1.67 | 56 | 35 | 66 | 66 | 1.00 |
| 14 | N/S | N/S | N/S | | 57 | 14 | 64 | 25 | 2.56 |
| 15 | 35 | 71 | 65 | 1.09 | 58 | 32 | 73 | 56 | 1.30 |
| 16 | N/S | N/S | N/S | | 59 | 60 | 60 | 120 | 0.50 |
| 17 | 35 | 111 | 52 | 2.13 | 60 | 34 | 65 | 59 | 1.10 |
| 19 | 33 | 69 | 60 | 1.15 | 62 | 60 | 99 | 114 | 0.87 |
| 20 | 20 | 96 | 46 | 2.09 | 63 | 40 | 73 | 64 | 1.14 |
| 21 | 32 | 70 | 65 | 1.08 | 64 | 26 | 68 | 51 | 1.33 |
| 22 | 20 | 74 | 55 | 1.35 | 74 | 28 | 81 | 51 | 1.59 |
| 23 | 40 | 166 | 89 | 1.87 | 75 | 40 | 62 | 64 | 0.97 |
| 24 | 28 | 86 | 78 | 1.10 | 76 | 28 | 76 | 51 | 1.49 |
| 26 | 23 | 81 | 40 | 2.03 | 78 | 60 | 74 | 97 | 0.76 |
| 28 | 22 | 65 | 57 | 1.14 | 80 | 29 | 68 | 47 | 1.45 |
| 29 | 20 | 116 | 83 | 1.40 | 81 | 37 | 70 | 72 | 0.97 |
| 30 | 36 | 72 | 64 | 1.13 | 82 | 40 | 72 | 49 | 1.47 |
| 31 | 20 | 84 | 65 | 1.29 | 83 | 40 | 73 | 71 | 1.03 |
| 33 | 30 | 63 | 53 | 1.19 | 85 | 28 | 60 | 41 | 1.46 |
| 34 | 14 | 92 | 36 | 2.56 | 86 | 29 | 78 | 50 | 1.56 |
| 35 | 20 | 94 | 39 | 2.41 | 87 | 28 | 63 | 50 | 1.26 |
| 36 | N/S | N/S | N/S | | 93 | 40 | 78 | 76 | 1.03 |
| 47 | 34 | 63 | 58 | 1.09 | 94 | 30 | 60 | 54 | 1.11 |
| 48 | 40 | 68 | 61 | 1.11 | 95 | 60 | 201 | 139 | 1.45 |
| 49 | 40 | 71 | 62 | 1.15 | 96 | 32 | 67 | 64 | 1.05 |
| 50 | 60 | 102 | 116 | 0.88 | 97 | 40 | 71 | 74 | 0.96 |
| 51 | 33 | 62 | 58 | 1.07 | 98 | 31 | 106 | 87 | 1.22 |
| 52 | 31 | 63 | 83 | 0.76 | 99 | 40 | 99 | 74 | 1.34 |
| 53 | 30 | 65 | 61 | 1.07 | 100 | 40 | 74 | 73 | 1.01 |
| 65 | 29 | 71 | 49 | 1.45 | 101 | 40 | 148 | 85 | 1.74 |
| 66 | 16 | 78 | 38 | 2.05 | 102 | 60 | 92 | 88 | 1.05 |
| 67 | 30 | 67 | 51 | 1.31 | 103 | N/S | N/S | N/S | |
| 68 | 28 | 71 | 60 | 1.18 | 104 | 30 | 93 | 86 | 1.08 |
| 69 | 22 | 72 | 39 | 1.85 | 105 | 40 | 98 | 84 | 1.17 |
| 70 | 30 | 97 | 61 | 1.59 | | | | | |
| 71 | 40 | 76 | 74 | 1.03 | | | | | |
| 72 | 60 | 70 | 98 | 0.71 | | | | | |
| 73 | 28 | 79 | 65 | 1.22 | | | | | |
| 92 | 34 | 66 | 63 | 1.05 | | | | | |
| 106 | 60 | 180 | 104 | 1.73 | | | | | |
| 107 | 20 | 78 | 50 | 1.56 | | | | | |
| 108 | 13 | 60 | 22 | 2.73 | | | | | |
| 109 | 18 | 70 | 35 | 2.00 | | | | | |

Conclusion: This study supports the notion that amplification of ESR1 might be a marker for tamoxifen resistance in patients with operable and ER positive breast cancer.

### Example 5

### Frequency of ESR1 Deletions and Amplifications in Patients Enrolled in the Clinical Trial DBCG 89D

Patients enrolled in the clinical trial DBCG 89D (Ejlertsen; 2007) have previously been tested for the prognostic and predictive value of TOP2A gene aberrations (Knoop; 2005). The patient cohort has a high frequency of ER negative patients and has thus been a well suited material for investigating the existence of ESR1 deletions and the relationship between ESR1 gene aberrations and ER protein as tested by IHC. The present study explores the relationship between ESR1 and ER in the DBCG 89-D trial.

Material and Methods: The DBCG 89-D trial randomized 962 high-risk Danish breast cancer patients to nine series of CMF or CEF, without endocrine therapy. Overall CEF was superior to CMF in terms of DFS and OS. TMA's were constructed and analyzed centrally for ER expression and ESR1 copy number changes using FISH. Relationships between biomarkers and DFS were analyzed using uni- and multivariate statistics.

Results: 667 blocks (69% of total eligible) have been collected and the ESR1 test was successful in 607 (91%). 8 patients (1%) had ESR1 amplification (ratio>2) and 162 (27%) had ESR1 deletion (ratio<0.8). ER expression was associated to (p<0.01) but not exclusively dependent on ESR1 aberrations. ESR1 deletion was not significantly associated with other established prognostic factors including positive nodes, tumor size, grade, HER2 or TOP2A (see Table 7 below).

TABLE 7
Patient Characteristics

| | Deletion | | Normal | | Amplified | |
|---|---|---|---|---|---|---|
| | (N = 162) | | (N = 437) | | (N = 8) | |
| | No. | % | No. | % | No. | % |
| Age at enrolment, years | | | | | | |
| <39 | 26 | 16 | 61 | 14 | 0 | — |
| 40-49 | 80 | 49 | 212 | 49 | 4 | 50 |
| 50-59 | 35 | 22 | 103 | 24 | 2 | 25 |
| 60-69 | 21 | 13 | 61 | 14 | 2 | 25 |
| Menopausal status | | | | | | |
| Premenopausal | 107 | 66 | 300 | 69 | 4 | 50 |
| Postmenopausal | 55 | 34 | 137 | 31 | 4 | 50 |
| Loco-regional therapy | | | | | | |
| Breast-conserving surgery | | | | | | |
| Mastectomy | | | | | | |
| Nodal status | | | | | | |
| 0 positive nodes | 54 | 33 | 154 | 35 | 3 | 38 |
| 1-3 positive nodes | 55 | 34 | 142 | 33 | 0 | 0 |
| ≧4 positive nodes | 53 | 33 | 141 | 32 | 5 | 63 |
| Tumor size | | | | | | |
| 0-20 mm | 60 | 37 | 177 | 41 | 1 | 13 |
| 21-50 mm | 86 | 53 | 222 | 51 | 7 | 87 |
| >50 mm | 15 | 9 | 37 | 8 | 0 | — |
| Unknown | 1 | 1 | 1 | — | 0 | — |
| Histologic type | | | | | | |
| Infiltrating ductal | 150 | (93) | 405 | (93) | 8 | (100) |
| carcinoma | | | | | | |
| Other carcinomas | 12 | (7) | 31 | (7) | 0 | (—) |
| Unknown | 0 | (—) | 1 | (—) | 0 | (—) |
| Malignancy grade (ductal | | | | | | |
| carcinoma only) | | | | | | |
| Grade I | 6 | (4) | 38 | (9) | 1 | (13) |
| Grade II | 74 | (49) | 191 | (48) | 3 | (38) |
| Grade III | 69 | (46) | 175 | (43) | 4 | (50) |
| Unknown | 1 | (1) | 1 | (3) | 0 | (—) |
| Hormone receptor | | | | | | |
| Positive | 40 | (25) | 160 | (37) | 3 | (38) |
| Negative | 121 | (75) | 271 | (62) | 5 | (63) |
| Unknown | 1 | (1) | 6 | (1) | 0 | (—) |

Conclusion: Deletions in ESR1 were present in a large group of predominantly ER negative patients in the DBCG 89D trial.

### REFERENCES

1. Ejlertsen B, Mouridsen H T, Jensen M B, Andersen J, Cold S, Edlund P, et al. Improved outcome from substituting methotrexate with epirubicin: Results from a randomised comparison of CMF versus CEF in patients with primary breast cancer. Eur J Cancer 2007; 43(5):877-84.
2. Knoop A S, Knudsen H, Balslev E, Rasmussen B B, Overgaard J, Nielsen K V, et al. retrospective analysis of topoisomerase IIa amplifications and deletions as predictive markers in primary breast cancer patients randomly assigned to cyclophosphamide, methotrexate, and fluorouracil or cyclophosphamide, epirubicin, and fluorouracil: Danish Breast Cancer Cooperative Group. J Clin Oncol 2005; 23(30):7483-90.

### Example 6

### Gene Copy Numbers of BCL2, FASN, SCUBE2, ESR2, PGR, BIRC5 and COX2 in Samples from Patients Treated with Tamoxifen

Material and methods: Tumor material was collected from 86 postmenopausal ER-positive breast cancer patients. The patients had primary operative breast cancer and were after surgery allocated five years of tamoxifen according to DBCG guidelines (DBCG 95-C). The patients were selected to fit two groups: one group was recurrence free after seven years from initiating the adjuvant tamoxifen treatment. The other group had disease recurrence, other malignant disease, or death within four years from initiation of tamoxifen therapy. The patients in the recurrence group had significantly higher number of positive lymph nodes (P=0.0003) and a higher malignancy grade (P=0.0009) than the patients in the non-recurrence group.

Paraffin-embedded tissue blocks were collected from the above mentioned patients and TMAs were constructed. Representative areas of invasive tumor cells from each patient were selected from corresponding hematoxylen and eosin (HE)-stained sections and TMAs were performed by inserting two 2 mm diameter cores from each patient into an empty block in an ordered manner. Two samples of kidney and liver tissue were integrated among the breast carcinoma tissues in each TMA as reference for orientation. Subsequently, 3 μm sections were cut of the TMA blocks onto adhesive-coated slides which were baked overnight at 65° C.

The tumor samples were analyzed for gene copy number using FISH. The FISH assay was performed according to the method described in Example 1 and in the Detailed Description of Invention.

Correlations between observed GCVs (Genomic Copy number Variants) and clinical outcome were performed with the use of Fischer's exact two-tailed tests, which allows for few observations. A value of P<0.05 was considered statistically significant. Patients for whom one or more gene status results were missing where excluded from the statistical analysis. The GCVs were analyzed in panels where a GCV (amplification/deletion) was defined as minimum one GCV in one of the target genes for a given patient. Gene amplifications and deletions were tested versus non-amplifications (normal added deletions) and non-deletions (normal added amplifications), respectively

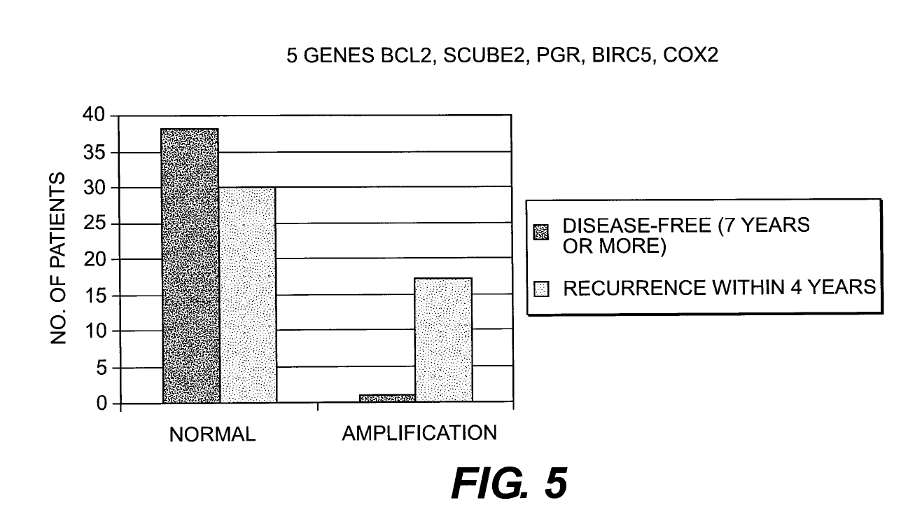

Copy number aberrations have been studied in five genes of the ESR1-related genes, i.e. BCL2, SCUBE2, PGR, BIRC5 and COX2 (results are presented in Table 8) and FIG. 5), using the patient cohort described in Example 4. Data on 86 patients.

The five genes have been combined into a profile (five genes panel I): BCL2, SCUBE2, PGR, BIRC5 and COX. The FISH analysis was successful in a total of 86 patient samples. The tamoxifen treated patients who had tumors containing amplification in any of the 5 genes had a worse outcome of treatment when compared to tamoxifen treated patients with tumors not containing any amplification in the 5 genes, the p-value being 0.0001.

TABLE 8
Five-genes profile: BCL2, SCUBE2, PGR, BIRC5, COX2

| | Normal | Amplification | Total |
|---|---|---|---|
| Disease-free (7 years or more) | 38 | 1 | 39 |
| Recurrence within 4 years | 30 | 17 | 47 |
| | | | 86 |

Fisher's exact test (two-tailed): P = 0.0001
X² (with Yates correction) = 12.585

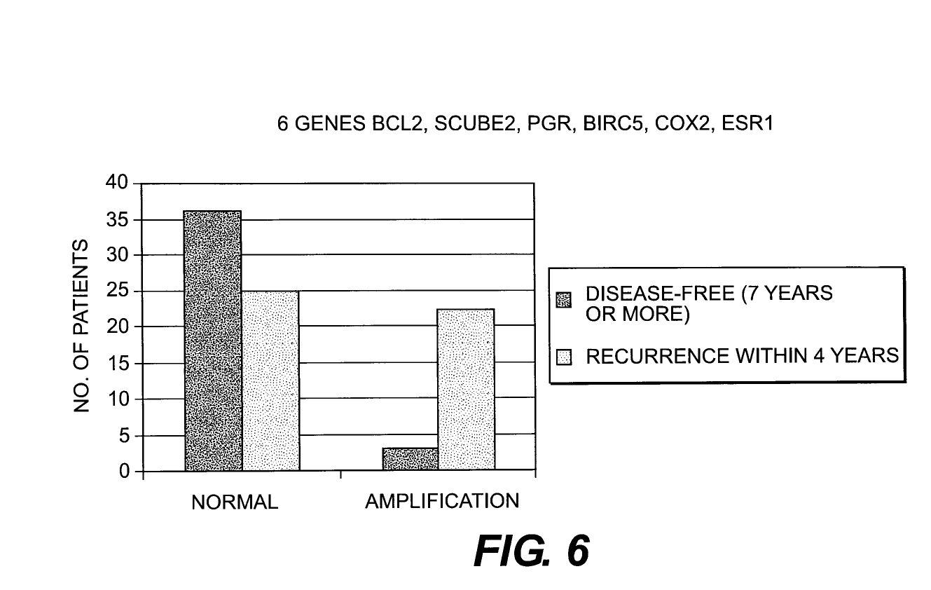

With the addition of ESR1 to the five genes profile, a six genes profile (classifier) consisting of BCL2, SCUBE2, PGR, BIRC5, COX and ESR1 was created and tested. The results of the tests are shown in Table 9 and FIG. 6. The tamoxifen treated patients who had tumors containing amplification in any of the 6 genes had a worse outcome of treatment as well when compared to tamoxifen treated patients with tumors not containing amplification in any of the 6 genes considered, the p-value being 0.0001.

TABLE 9
Six-gene profile: ESR1, BCL2, SCUBE2, PGR, BIRC5, COX2

| | Normal | Amplification | Total |
|---|---|---|---|
| Disease-free (7 years or more) | 36 | 3 | 39 |
| Recurrence within 4 years | 25 | 22 | 47 |
| | | | 86 |

Fisher's exact test (two-tailed): P = 0.0001
X² (with Yates correction) = 13.976

Sensitivity of the tests (the proportion of positives that are correctly identified by the test according to Altman, D. G. 1991 (Statistics for Medical Research, Chapman & Hall). Sensitivity of the gene panel is 53%. Specificity (the proportion of negatives that are correctly identified by the test) is 86%

### Example 7

### A Seven-Gene Profile: Estimation of Copy Numbers of ESR1, BCL2, FASN, SCUBE2, PGR, BIRC5 and ESR21N Samples from Patients Treated with Tamoxifen has a Predictive Value for the Outcome of Treatment

Six of the ESR1-related genes ESR2, PGR, SCUBE2, BCL2, BIRC5, and FASN were tested individually for different distribution of gene status in the two recurrence groups. No significant difference was found for any of the genes. The genes were also tested individually for differences in the number of deletions in the non-recurrence versus the recurrence group of patients. No significant difference was found for any of the seven genes. The six genes were also tested as a panel of seven genes including of ESR1, ESR2, PGR, SCUBE2, BCL2, BIRC5, and FASN (n=79). The experiments were performed as described in EXAMPLE 5.

The seven genes were tested individually for different distribution of gene amplifications in the two recurrence groups. The recurrence group had significant more BIRC5 amplifications than the patients in the non-recurrence group (P=0.032). No significant difference was observed between the two groups in the number of amplifications for any of the six other genes ESR1, ESR2, PGR, SCUBE2, BCL2, and FASN. The panel of seven genes showed no difference between the number of deletions observed in the non-recurrence group (n=21) versus the recurrence group (n=29).

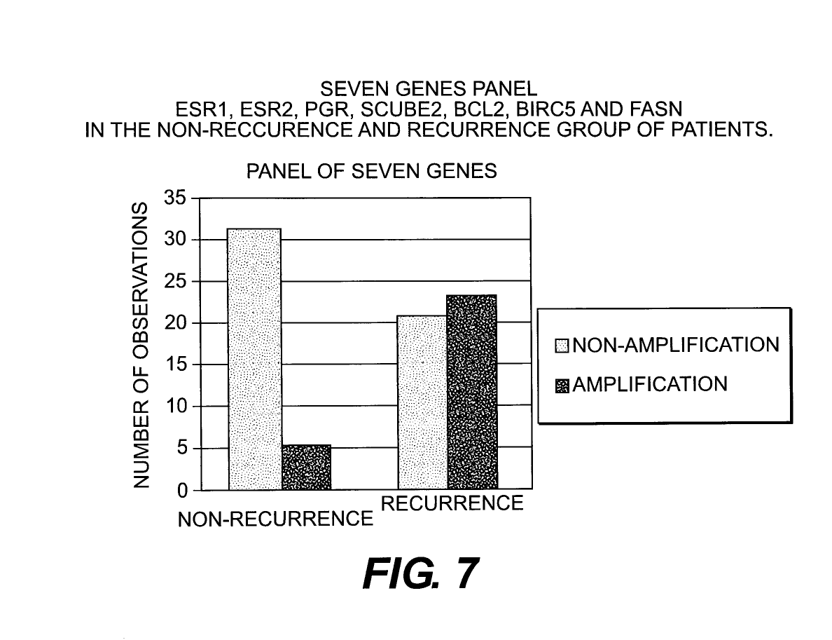

The seven gene panel of ESR1, ESR2, PGR, SCUBE2, BCL2, BIRC5, and FASN was tested for dissimilar distribution of gene amplifications in the two recurrence groups; see Table 11 and 12 and FIG. 7. The patients in the recurrence group had significant more amplifications in all genes of the seven genes panel, than the patients in the non-recurrence group (P=0.0003)

Table 10 summarizes the gene status of ESR1, ESR2, PGR, SCUBE2, BCL2, BIRC5, and FASN in the breast cancer patients. Gene status is given in total and divided in the non-recurrence and recurrence group. The percentage of the relative gene status of a given group in total is given to the right of each observation.

TABLE 10

| | | ESR1 | | ESR2 | | PGR | | SCUBE2 | | BCL2 | | BIRC5 | | FASN | |
|---|---|---|---|---|---|---|---|---|---|---|---|---|---|---|---|
| Total | Normal | 68 | 82% | 68 | 82% | 49 | 59% | 71 | 85% | 55 | 66% | 73 | 88% | 77 | 92% |
| | Deletion | 4 | 5% | 9 | 11% | 31 | 37% | 9 | 11% | 21 | 25% | 4 | 5% | 3 | 4% |
| | Amplification | 11 | 13% | 4 | 5% | 2 | 2% | 3 | 4% | 7 | 8% | 6 | 7% | 2 | 2% |
| | Missing | 0 | — | 2 | 3% | 1 | 1% | 0 | — | 0 | — | 0 | — | 1 | 1% |
| | Total | 83 | | 83 | | 83 | | 83 | | 83 | | 83 | | 83 | |
| Non-recurrence | Normal | 32 | 89% | 30 | 81% | 24 | 67% | 34 | 94% | 25 | 70% | 35 | 97% | 35 | 97% |
| | Deletion | 2 | 6% | 4 | 11% | 12 | 33% | 2 | 6% | 10 | 28% | 1 | 3% | 1 | 3% |
| | Amplification | 2 | 6% | 2 | 6% | 0 | — | 0 | — | 1 | 3% | 0 | — | 0 | — |
| | Missing | 0 | — | 0 | — | 0 | — | 0 | — | 0 | — | 0 | — | 0 | — |
| | Total | 36 | | 36 | | 36 | | 36 | | 36 | | 36 | | 36 | |
| Recurrence | Normal | 36 | 77% | 38 | 81% | 25 | 52% | 37 | 79% | 30 | 64% | 38 | 81% | 42 | 89% |
| | Deletion | 2 | 4% | 5 | 11% | 19 | 40% | 7 | 15% | 11 | 23% | 3 | 6% | 2 | 4% |
| | Amplification | 9 | 19% | 2 | 4% | 2 | 4% | 3 | 6% | 6 | 13% | 6 | 13% | 2 | 4% |
| | Missing | 0 | — | 2 | 4% | 1 | 2% | 0 | — | 0 | — | 0 | — | 1 | 2% |
| | Total | 47 | | 47 | | 47 | | 47 | | 47 | | 47 | | 47 | |

Table 11 summarizes data on non-amplification and amplification of the genes of the seven gene panel consisting of ESR1, ESR2, PGR, SCUBE2, BCL2, BIRC5, and FASN in the non-recurrence and recurrence group of patients.

TABLE 11

| | Non-amplification | Amplification | Total |
|---|---|---|---|
| Non-recurrence | 31 | 5 | 36 |
| Recurrence | 20 | 23 | 43 |
| Total | 51 | 28 | 79 |

Sensitivity of the seven genes panel is 53%, specificity of the panel is 86%.

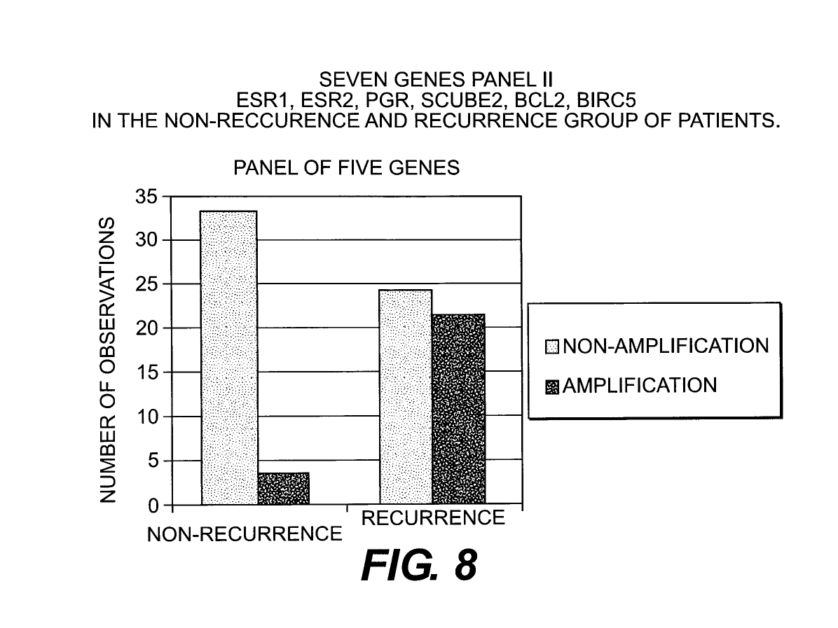

To acquire a higher sensitivity ESR2 and FASN were excluded and a five gene panel II consisting of ESR1, PGR, SCUBE2, BCL2, and BIRC5 was constructed (n=82), see Table 12 and FIG. 8.

Table 12 summarizes data on detected non-amplifications and amplifications in the genes of the five gene panel II, namely ESR1, PGR, SCUBE2, BCL2, and BIRC5, in the non-recurrence and recurrence group of patients.

TABLE 12

| | Non-amplification | Amplification | Total |
|---|---|---|---|
| Non-recurrence | 33 | 3 | 36 |
| Recurrence | 24 | 22 | 46 |
| Total | 57 | 25 | 82 |

The recurrence group of breast cancer patients had significant more amplifications in the genes of the five gene panel II (ESR1, PGR, SCUBE2, BCL2, and BIRC5) compared to the number of amplifications in the genes in the non-recurrence group (P=0.0001). The sensitivity of the five gene panel II is 48% and the specificity is 92%.

### Example 8

### Amplification of the Genes of a Four Genes Panel, ESR1, SCUBE2, BCL2 and BIRC5, is Predictive for the Outcome of Tamoxifen Treatment

Material and Methods: Within a consecutive series of postmenopausal patients allocated to tamoxifen 20 mg daily for 5 years following radical surgery for early hormone receptor positive breast cancer, we identified 61 patients with recurrence less than 4 years and 48 patients with recurrence more than 7 years after initiation of adjuvant tamoxifen.

Archival tissue from the primary tumor was collected from 100 of the 109 patients (92%). The tumor samples were analyzed for copy number changes using FISH with probes covering the each gene and a reference probe covering the centromere of the particular chromosome. FISH was performed with Dako Histology FISH accessory kit.

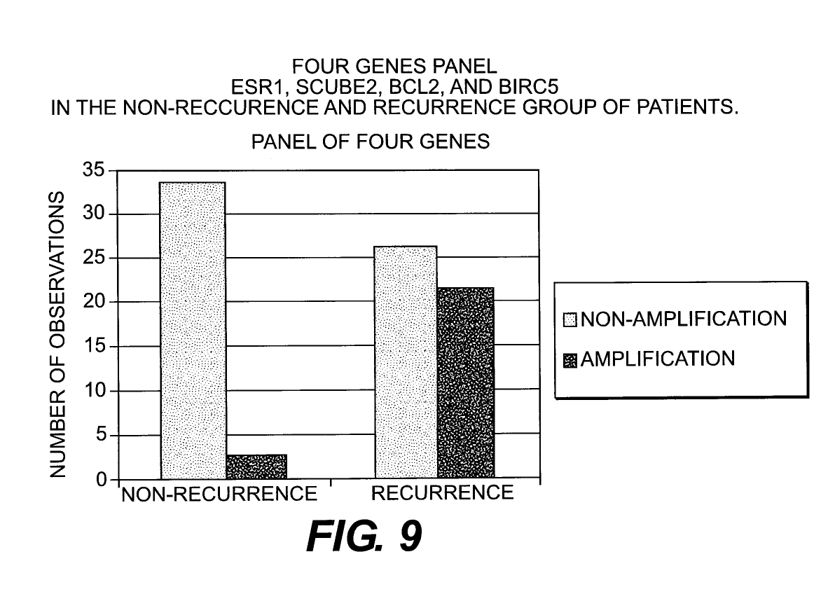

Results: The FISH analysis for all 4 genes was successful in 83 of the 100 patients (83%). Amplification (ratio gene/CEN≧2) was observed in 21 of 47 (45%) patients with recurrence earlier than 4 years, compared to 3 of 36 (8%) patients who were free of recurrence for more than 7 years (p=0.0002). In both groups, patients with deletions (ratio gene/CEN<0.8) were also identified. Summarized results of the study evaluated the number of cases having normal and amplified genes of the panel ESR1, SCUBE2, BCL2, and BIRC5 in the non-recurrence and recurrence group of patients are presented in Table 13 below and FIG. 9.

Table 13 summarizes data on detected non-amplifications and amplifications in the genes of the four genes panel II, namely ESR1, SCUBE2, BCL2, and BIRC5, in the non-recurrence and recurrence group of patients

TABLE 13

| | Non-amplification | Amplification | Total |
|---|---|---|---|
| Non-recurrence | 33 | 3 | 36 |
| Recurrence | 26 | 21 | 47 |
| Total | 59 | 24 | 83 |

Discussion: This study demonstrates that amplification of four genes including ESR1 and three genes selected from the group of ESR1-related genes of the invention, namely SCUBE2, BCL2 or BIRC5 may serve as an indicator of tamoxifen resistance in patients with operable and ER positive breast cancer and used as a prognostic marker for the outcome of hormone therapy treatment. The study also revealed the presence of deletions of these genes in patients. Use the latter status of the genes this panel as a prognostic and predictive factor of in connection with estrogen treatment is also possible.

1. A method for predicting the efficacy of a therapeutic treatment of a cancer patient comprising
determining the status of aberration of the ESR1 gene and, optionally, the status of aberration of at least one ESR1-related gene in a sample obtained from said patient;
predicting the efficacy of a therapeutic treatment based on the determined status of aberration of the ESR1 gene and, optionally, on the determined status of aberration of the at least one ESR1-related gene.

2. The method of claim 1, wherein the therapeutic treatment is selected from anti-cancer hormone therapy or chemotherapy.

3. The method of claim 1, wherein the cancer patient is a patient who has or who is suspected of having a breast, ovarian, prostate, cervical, corpus uteri cancer or endometrial carcinoma.

4. The method of claim 1, wherein the sample is a tissue sample.

5. The method of claim 4, wherein the tissue sample is a biopsy, frozen tissue section, paraffin embedded tissue section, smear, exudates, ascites, blood, bone marrow, sputum, urine or any tissue treated with a fixative.

6. The method of claim 1, wherein the ESR1-related gene is selected from the ESR2, PGR, SCUBE2, BCL2, BIRC5, FASN or COX genes.

7. The method of claim 1, wherein the aberration is amplification, duplication, polyploidization, deletion or translocation of said gene(s), part(s) of said gene(s), or part(s) of the chromosome(s) comprising a nucleic sequence(s) controlling the expression of the gene(s).

8. The method of claim 7, wherein the status of aberration is determined as the presence or absence of aberration.

9. The method of claim 8, wherein the status of the aberration is determined by a method comprising a step of in situ hybridization analysis of the tissue sample in vitro.

10. The method of claim 9, wherein the in situ hybridization analysis is selected form a Flourescent In Situ Hybridization (FISH) or Chromogen In Situ Hybridization analysis (CISH).

11. The method of claim 10, wherein the in situ hybridization analysis comprises using at least one probe targeted at the ESR1 gene region and at least one reference probe.

12. The method of claim 11, wherein the reference probe is targeted at the centromere region.

13. The method of claim 12, wherein the at least one reference probe is targeted at the centromere region of chromosome 6.

14. The method of claim 11, wherein at least two different gene targeted probes are used.

15. The method of claim 14, wherein at least one probe is targeted at the ESR1 gene region and at least one gene targeted probe is targeted at an ESR1-related gene.

16. The method according to claim 11, wherein the gene targeted and reference probes comprise labels and the label of the gene target probe can be distinguished from the label of the reference probe.

17. The method according to claim 8, wherein the presence of amplification of ESR1 in a patient sample is correlated with the hormone therapy resistance and high likelihood of recurrence of the disease in the patient.

18. The method according to claim 1, wherein the method comprising determining the status of aberration of ESR1 and the status of aberration of one or more the ESR1-related genes.

19. The method according to claim 18, wherein the presence of aberration in the one or more ESR1-releted genes in a patient sample is correlated with the hormone therapy resistance and high likelihood of recurrence of the disease in the patient.

20. A method for selecting a therapeutic treatment for a cancer patient comprising
determining the status of aberration of the ESR1 gene and, optionally, the status of aberration of at least one ESR1-related gene in a sample obtained from said patient;
selecting a therapeutic treatment based on the determined status of aberration of the ESR1 gene and, optionally, on the determined status of aberration of the at least one ESR1-related gene.

21. A method for stratifying cancer patients for different therapeutic treatments comprising
determining a status of aberration of the ESR1 gene in samples obtained from the cancer patients;
stratifying the cancer patients for different therapeutic treatments based on the determined status of aberration of the ESR1 gene and, optionally, on the determined status of aberration of the at least one ESR1-related gene.

22. A method for prognosis of likelihood of recurrence of cancer in a cancer patient who was or is under a course of therapeutic treatment, comprising
determining the status of aberration of the ESR1 gene and, optionally, the status of aberration of at least one ESR1-related gene in a sample obtained from said patient;
predicting the likelihood of recurrence of the cancer in the patient based on the determined status of aberration of the ESR1 gene and, optionally, on the determined status of aberration of the at least one ESR1-related gene.

23. A kit comprising at least two different probes for in situ hybridization, comprising at least one probe targeted at the ESR1 gene, or at a part of said gene, and at least one reference probe.
